SEQUENCE LISTING (51 sequences)

SEQ ID NO: 1 - DNA, 426 bp, Cauliflower mosaic virus
  source 1..426: mol_type = genomic DNA
gtcaacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggacacgc

SEQ ID NO: 2 - DNA, 62 bp, Tobacco mosaic virus
  source 1..62: mol_type = genomic DNA
atttttacaacaattaccaacaacaacaaacaacaaacaacattacaattacatttacaatt

SEQ ID NO: 3 - DNA, 263 bp, Agrobacterium tumefaciens
  source 1..263: mol_type = genomic DNA
gtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcga

SEQ ID NO: 4 - DNA, 714 bp, Agrobacterium tumefaciens
  source 1..714: mol_type = genomic DNA
gtcctgctttaatgagatatgcgagaagcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggac

SEQ ID NO: 5 - DNA, 1158 bp, synthetic construct
  source 1..1158: mol_type = other DNA; note = Protein sequence for chicken ovalbumin was taken from the Gallus gallus genome and condon optimized for expression in soybean, free of introns.
atggggagtatcggggcagcaagcatggagttttgtttcgacgtctttaaggaacttaaagtccaccatgctaatgaaaatatattttattgccccatcgcaatcatgtctgctttggccatggtttatctgggggccaaggattccaccagaacccagatcaataaagtggtcaggttcgacaaactgcctggattcggggatagcatagaagctcagtgtggcacatcagtgaatgttcattcttctttgagggacatcctgaaccaaatcacaaaacccaacgatgtgtatagtttttcactggctagtcgtctttacgctgaggagaggtacccaattttgcccgagtacctgcaatgtgtaaaagagttgtacagaggaggtttggaacctataaacttccaaaccgcagcagatcaggctcgcgaactcatcaacagttgggttgagagtcaaacaaatggtattatcaggaatgtactccagccatcaagcgtggactcacaaaccgctatggtcctcgtaaatgcaattgtattcaaagggttgtgggagaaggcttttaaagacgaggatacacaggctatgcctttccgcgttactgaacaagaatccaaaccagtacaaatgatgtatcaaatcggactctttcgcgtcgccagcatggcctcagaaaagatgaagatcctcgagctgccattcgcatcagggactatgagcatgctcgtactcttgccagacgaagtttcaggattggaacagcttgagagtataataaattttgagaagctgactgagtggaccagttctaacgttatggaggaaaggaaaattaaagtttaccttcccagaatgaaaatggaagaaaagtacaatctcaccagtgttcttatggctatggggataacagatgtcttttcctcaagcgccaacttgagcggcatatctagtgctgaatccttgaagattagtcaagccgtccatgctgctcatgcagagatcaatgaggcaggccgtgaagttgtgggcagtgctgaagctggggtagatgcagccagtgtctctgaggagttccgtgccgaccatcctttcttgttctgcatcaagcatatcgccacaaacgcagtactcttcttcggtaggtgcgtgagtcct

SEQ ID NO: 6 - DNA, 558 bp, synthetic construct
  source 1..558: mol_type = other DNA; note = Protein sequence for chicken ovomucoid was taken from the Gallus gallus genome and condon optimized for expression in soybean, free of introns.
gccgaagttgattgttcaagattccccaatgccacagataaagaaggcaaagacgttttggtctgcaacaaggaccttcgccctatttgcggcactgacggggtcacatataccaatgactgtctgttgtgcgcctactccatagaatttgggactaacatatccaaggagcacgatggggaatgcaaagagacagtccccatgaattgctcctcctatgcaaataccactagtgaagatggcaaggtaatggtactctgtaacagggctttcaatccagtgtgtggcactgatggtgttacctatgacaatgaatgtctcttgtgcgcacataaggtggaacaaggtgcctccgtagacaagcgccacgacggaggttgcagaaaggaactcgccgctgtttcagtggattgctccgagtaccctaagcccgattgcacagcagaagatcgtccattgtgtggaagcgataacaagacttatggcaataagtgcaatttctgtaacgccgtagtggagtcaaatgggacccttaccctcagtcactttgggaaatgt

SEQ ID NO: 7 - DNA, 57 bp, Glycine max
  source 1..57: mol_type = genomic DNA
atggccaagctagttttttccctttgttttctgcttttcagtggctgctgcttcgct

SEQ ID NO: 8 - DNA, 63 bp, Arabidopsis thaliana
  source 1..63: mol_type = genomic DNA
atggcaaacaagctcttcctcgtctgcgcaactttcgccctctgcttcctcctcaccaacgct

SEQ ID NO: 9 - DNA, 55 bp, Glycine max
  source 1..55: mol_type = genomic DNA
atcaagaaccagttcaatatcctagtacaccgtattaaagaatttaagatatact

SEQ ID NO: 10 - DNA, 12 bp, synthetic construct
  source 1..12: mol_type = other DNA; note = Codon optimized, originally from Arabidopsis thaliana.
catgatgagttg

SEQ ID NO: 11 - DNA, 12 bp, synthetic construct
  source 1..12: mol_type = other DNA; note = Codon optimized, originally from Arabidopsis thaliana
aaggacgaactc

SEQ ID NO: 12 - DNA, 69 bp, Glycine max
  source 1..69: mol_type = genomic DNA
atggggaagcccttcactctctctctttcttccctttgcttgctactcttgtcgagtgcatgctttgct

SEQ ID NO: 13 - DNA, 12 bp, synthetic construct
  source 1..12: mol_type = other DNA; note = Codon optimized for expression in soybean. Codes for 4x glycine.
ggaggaggagga

SEQ ID NO: 14 - DNA, 12 bp, synthetic construct
  source 1..12: mol_type = other DNA; note = Codon optimized for expression in soybean. Codes for 4x lysine.
aagaagaagaag

SEQ ID NO: 15 - DNA, 72 bp, Glycine max
  source 1..72: mol_type = genomic DNA
atggggaagcccttcttcactctctctctttcttccctttgcttgctactcttgtcgagtgcatgctttgct

SEQ ID NO: 16 - DNA, 30 bp, synthetic construct
  source 1..30: mol_type = other DNA; note = Codon optimized for expression in soybean. Codes for PLSSILRAFY peptide.
ccactttcttctattcttagagctttctat

SEQ ID NO: 17 - DNA, 45 bp, Hordeum vulgare
  source 1..45: mol_type = genomic DNA
gtttttgctgaggctattgctgctaactctactcttgttgctgag

SEQ ID NO: 18 - DNA, 45 bp, synthetic construct
  source 1..45: mol_type = other DNA; note = Protein sequence for Simian Virus 40 nuclear localization signal was taken from the Simian Virus 40 genome and condon optimized for expression in soybean.
atggcttctagcccaccgaagaagaagcggaaggtcagctggaaa

SEQ ID NO: 19 - DNA, 24 bp, synthetic construct
  source 1..24: mol_type = other DNA; note = Codon optimized for expression in soybean. Codes for DYKDDDDK peptide.
gattacaaagatgacgacgataag

SEQ ID NO: 20 - DNA, 27 bp, synthetic construct
  source 1..27: mol_type = other DNA; note = Codon optimized for expression in soybean. Codes for YPYDVPDYA peptide.
tacccctacgacgttcctgactacgcc

SEQ ID NO: 21 - DNA, 13 bp, synthetic construct
  source 1..13: mol_type = other DNA; note = Concensus kozak sequence for translation initiation.
gccgccrccatgg

SEQ ID NO: 22 - DNA, 321 bp, Escherichia coli
  source 1..321: mol_type = genomic DNA
atgaccatgattacgccaagcttgcatgcctgcaggtcgactctagaggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgca

SEQ ID NO: 23 - DNA, 792 bp, synthetic construct
  source 1..792: mol_type = other DNA; note = DNA sequence for the neomycin phosphotransferase II enzyme was taken from the Echerichia coli O157:H7 strain ATCC 43890 genome and was modified to remove certain restriction sites for ease of molecular cloning.
atggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaggacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgctcagggaggatatcggggaagaacagtatgtcgagctattttttgacttactggggatcaagcctgattgggagaaaataaaatattatattttactggatgaattgttt

SEQ ID NO: 24 - DNA, 790 bp, synthetic construct
  source 1..790: mol_type = other DNA; note = DNA sequence for the pUC origin of replication was taken from the Escherichia coli strain NCTC12652 genome and modified to removed certain restriction sites for ease of molecular cloning.
cagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggc

SEQ ID NO: 25 - DNA, 618 bp, Escherichia coli
  source 1..618: mol_type = genomic DNA
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccgg

SEQ ID NO: 26 - DNA, 1149 bp, Escherichia coli
  source 1..1149: mol_type = genomic DNA
atgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtcttgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctag

SEQ ID NO: 27 - DNA, 151 bp, Agrobacterium tumefaciens
  source 1..151: mol_type = genomic DNA
tgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggt

SEQ ID NO: 28 - protein, 386 aa, Gallus gallus
MGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSP

SEQ ID NO: 29 - protein, 186 aa, Gallus gallus
AEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 30 - DNA, 4280 bp, synthetic construct
  source 1..4280: mol_type = other DNA; note = Synthetic plasmid vector for Agrobacterium-mediated transformation into plant cells.
cgctatgggacacgaagtgatccgtttaaactatcagtgtttgacaggatatattggcgggtaaacctaagagaaaagagcgtttattagaataatcggatatttaaaagggcgtgaaaaggtttatccgttcgtccatttgtatgtgccagccgcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggccaattcccgagtgcgcggaacccctatttgtttatttttctaaatacattcaaatatgtatccgctcatgagacaataaccctgataaatgcttcaataatattgaaaaaggaagagtatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaggacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgctcagggaggatatcggggaagaacagtatgtcgagctattttttgacttactggggatcaagcctgattgggagaaaataaaatattatattttactggatgaattgttttagctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggcagatcctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtcttgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagcccctgctcggatctgttggaccggacagtagtcatggttgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggttgaacactctgtgccaattcgcatgc

SEQ ID NO: 31 - DNA, 640 bp, synthetic construct
  source 1..640: mol_type = other DNA; note = Synthetic sequence cloned into pMOZ backbone.
tgaacactctgtgccaattcgcatgcggagtgagaccgcagctggcacgacaggtttgccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgacggtctcacgctatgggacacgaagt

SEQ ID NO: 32 - DNA, 7068 bp, synthetic construct
  source 1..7068: mol_type = other DNA; note = Syntheic sequence for expressing ovalbumin protein in plant cells.
gtgccaattcgcatgcggaggtcaacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggacacgctcgagtataagagctcatttttacaacaattaccaacaacaacaaacaacaaacaacattacaattacatttacaattatcgatacaatggccaagctagttttttccctttgttttctgcttttcagtggctgctgcttcgctatggggagtatcggggcagcaagcatggagttttgtttcgacgtctttaaggaacttaaagtccaccatgctaatgaaaatatattttattgccccatcgcaatcatgtctgctttggccatggtttatctgggggccaaggattccaccagaacccagatcaataaagtggtcaggttcgacaaactgcctggattcggggatagcatagaagctcagtgtggcacatcagtgaatgttcattcttctttgagggacatcctgaaccaaatcacaaaacccaacgatgtgtatagtttttcactggctagtcgtctttacgctgaggagaggtacccaattttgcccgagtacctgcaatgtgtaaaagagttgtacagaggaggtttggaacctataaacttccaaaccgcagcagatcaggctcgcgaactcatcaacagttgggttgagagtcaaacaaatggtattatcaggaatgtactccagccatcaagcgtggactcacaaaccgctatggtcctcgtaaatgcaattgtattcaaagggttgtgggagaaggcttttaaagacgaggatacacaggctatgcctttccgcgttactgaacaagaatccaaaccagtacaaatgatgtatcaaatcggactctttcgcgtcgccagcatggcctcagaaaagatgaagatcctcgagctgccattcgcatcagggactatgagcatgctcgtactcttgccagacgaagtttcaggattggaacagcttgagagtataataaattttgagaagctgactgagtggaccagttctaacgttatggaggaaaggaaaattaaagtttaccttcccagaatgaaaatggaagaaaagtacaatctcaccagtgttcttatggctatggggataacagatgtcttttcctcaagcgccaacttgagcggcatatctagtgctgaatccttgaagattagtcaagccgtccatgctgctcatgcagagatcaatgaggcaggccgtgaagttgtgggcagtgctgaagctggggtagatgcagccagtgtctctgaggagttccgtgccgaccatcctttcttgttctgcatcaagcatatcgccacaaacgcagtactcttcttcggtaggtgcgtgagtccttcgggtggtggaggaagtggtggaggaggtagtatggtttccaaaggggaagagctctttacaggagtagtacctattcttgtcgaactcgatggggatgtaaatggacacaaattctccgtgtctggcgagggtgagggagacgccacctatgggaaattgacactcaagtttatctgcaccaccggtaaattacccgtcccttggcccacattagttactacgttttcctacggcgtgcagtgtttcagtaggtaccctgaccatatgaagcaacatgacttcttcaagagcgcaatgcctgaaggatatgtgcaggagcgtacaattttctttaaagatgatggcaactacaagactagggccgaggttaagtttgaaggcgatactttggtaaataggattgagctcaagggtatcgacttcaaggaagatggcaacattttaggacacaagctcgaatacaattataattctcataacgtgtatatcatggctgataagcagaagaacggcatcaaagtgaacttcaagatccgtcataacattgaggatggctcagttcaactggcagatcattaccagcaaaatacgccaatcggcgatggacctgtcttacttcctgataatcactacctgtcaacgcagtcagcattgtcaaaagatcctaatgagaagagagatcacatggtgctccttgagttcgtgaccgccgctggcatcactcacggtatggatgagctgtataaagattacaaagatgacgacgataagcatgatgaactctaggcttgtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgacgctatgggacacgaagtgatccgtttaaactatcagtgtttgacaggatatattggcgggtaaacctaagagaaaagagcgtttattagaataatcggatatttaaaagggcgtgaaaaggtttatccgttcgtccatttgtatgtgccagccgcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggccaattcccgagtgcgcggaacccctatttgtttatttttctaaatacattcaaatatgtatccgctcatgagacaataaccctgataaatgcttcaataatattgaaaaaggaagagtatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaggacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgctcagggaggatatcggggaagaacagtatgtcgagctattttttgacttactggggatcaagcctgattgggagaaaataaaatattatattttactggatgaattgttttagctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggcagatcctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtcttgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagcccctgctcggatctgttggaccggacagtagtcatggttgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggttgaacactct

SEQ ID NO: 33 - DNA, 6468 bp, synthetic construct
  source 1..6468: mol_type = other DNA; note = Synthetic sequence for expression ovomucoid protein in plant cells.
gtgccaattcgcatgcggaggtcaacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggacacgctcgagtataagagctcatttttacaacaattaccaacaacaacaaacaacaaacaacattacaattacatttacaattatcgatacaatggccaagctagttttttccctttgttttctgcttttcagtggctgctgcttcgctgccgaagttgattgttcaagattccccaatgccacagataaagaaggcaaagacgttttggtctgcaacaaggaccttcgccctatttgcggcactgacggggtcacatataccaatgactgtctgttgtgcgcctactccatagaatttgggactaacatatccaaggagcacgatggggaatgcaaagagacagtccccatgaattgctcctcctatgcaaataccactagtgaagatggcaaggtaatggtactctgtaacagggctttcaatccagtgtgtggcactgatggtgttacctatgacaatgaatgtctcttgtgcgcacataaggtggaacaaggtgcctccgtagacaagcgccacgacggaggttgcagaaaggaactcgccgctgtttcagtggattgctccgagtaccctaagcccgattgcacagcagaagatcgtccattgtgtggaagcgataacaagacttatggcaataagtgcaatttctgtaacgccgtagtggagtcaaatgggacccttaccctcagtcactttgggaaatgttcgggtggtggaggaagtggtggaggaggtagtatggtttccaaaggggaagagctctttacaggagtagtacctattcttgtcgaactcgatggggatgtaaatggacacaaattctccgtgtctggcgagggtgagggagacgccacctatgggaaattgacactcaagtttatctgcaccaccggtaaattacccgtcccttggcccacattagttactacgttttcctacggcgtgcagtgtttcagtaggtaccctgaccatatgaagcaacatgacttcttcaagagcgcaatgcctgaaggatatgtgcaggagcgtacaattttctttaaagatgatggcaactacaagactagggccgaggttaagtttgaaggcgatactttggtaaataggattgagctcaagggtatcgacttcaaggaagatggcaacattttaggacacaagctcgaatacaattataattctcataacgtgtatatcatggctgataagcagaagaacggcatcaaagtgaacttcaagatccgtcataacattgaggatggctcagttcaactggcagatcattaccagcaaaatacgccaatcggcgatggacctgtcttacttcctgataatcactacctgtcaacgcagtcagcattgtcaaaagatcctaatgagaagagagatcacatggtgctccttgagttcgtgaccgccgctggcatcactcacggtatggatgagctgtataaagattacaaagatgacgacgataagcatgatgaactctaggcttgtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgacgctatgggacacgaagtgatccgtttaaactatcagtgtttgacaggatatattggcgggtaaacctaagagaaaagagcgtttattagaataatcggatatttaaaagggcgtgaaaaggtttatccgttcgtccatttgtatgtgccagccgcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggccaattcccgagtgcgcggaacccctatttgtttatttttctaaatacattcaaatatgtatccgctcatgagacaataaccctgataaatgcttcaataatattgaaaaaggaagagtatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaggacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgctcagggaggatatcggggaagaacagtatgtcgagctattttttgacttactggggatcaagcctgattgggagaaaataaaatattatattttactggatgaattgttttagctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggcagatcctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtcttgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagcccctgctcggatctgttggaccggacagtagtcatggttgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggttgaacactct

SEQ ID NO: 34 - DNA, 6922 bp, synthetic construct
  source 1..6922: mol_type = other DNA; note = Synthetic sequence for expressing ovomucoid fused to moxBFP protein in plant cells.
gtgccaattcgcatgcggaggtcaacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggacacgctcgagtataagagctcatttttacaacaattaccaacaacaacaaacaacaaacaacattacaattacatttacaattatcgatacaatggccaagctagttttttccctttgttttctgcttttcagtggctgctgcttcgctgccgaagttgattgttcaagattccccaatgccacagataaagaaggcaaagacgttttggtctgcaacaaggaccttcgccctatttgcggcactgacggggtcacatataccaatgactgtctgttgtgcgcctactccatagaatttgggactaacatatccaaggagcacgatggggaatgcaaagagacagtccccatgaattgctcctcctatgcaaataccactagtgaagatggcaaggtaatggtactctgtaacagggctttcaatccagtgtgtggcactgatggtgttacctatgacaatgaatgtctcttgtgcgcacataaggtggaacaaggtgcctccgtagacaagcgccacgacggaggttgcagaaaggaactcgccgctgtttcagtggattgctccgagtaccctaagcccgattgcacagcagaagatcgtccattgtgtggaagcgataacaagacttatggcaataagtgcaatttctgtaacgccgtagtggagtcaaatgggacccttaccctcagtcactttgggaaatgttcgggtggtggaggaagtggtggaggaggtagtatggtgtctaagggggaagaactgtttaccggggttgtacctattttggtcgaattggacggagacgtcaatggtcataaatttagcgtgcgaggcgaaggagaaggggatgcaactaatgggaaactgactcttaaatttatcagcacgacagggaaattgcctgtgccctggcctacattggttactacgttatcacacggagttcaagttttcgccagataccccgatcatatgaagcaacatgatttcttcaagagcgccatgcccgaaggttatgtccaggagcgtacgatcttcttcaaagatgacgggacatacaaaacgagggctgaagtcaaatttgaaggcgatacgttagtcaacagaatcgaactgaaaggtgtcgactttaaggaggatggaaatattctgggtcataagttggaatataattttaattcccataacatttacattatggctgttaaacagaaaaacgggataaaggctaattttaagatcaggcacaatgtagaggacggttctgtacagcttgccgaccactaccagcaaaatactcccattggagacggaccagtgttattaccagatagccattacctttcaacacagagtaaattaagcaaagatccgaacgagaagcgagatcatatggtacttctcgaattcaggaccgcagcaggtataacgctcgggatggatgaactctataagtacccctacgacgttcctgactacgcccacgacgagctttaggcttgtcctgctttaatgagatatgcgagaagcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggaccgctatgggacacgaagtgatccgtttaaactatcagtgtttgacaggatatattggcgggtaaacctaagagaaaagagcgtttattagaataatcggatatttaaaagggcgtgaaaaggtttatccgttcgtccatttgtatgtgccagccgcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggccaattcccgagtgcgcggaacccctatttgtttatttttctaaatacattcaaatatgtatccgctcatgagacaataaccctgataaatgcttcaataatattgaaaaaggaagagtatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaggacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgctcagggaggatatcggggaagaacagtatgtcgagctattttttgacttactggggatcaagcctgattgggagaaaataaaatattatattttactggatgaattgttttagctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggcagatcctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtcttgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagcccctgctcggatctgttggaccggacagtagtcatggttgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggttgaacactct

SEQ ID NO: 35 - DNA, 7285 bp, synthetic construct
  source 1..7285: mol_type = other DNA; note = Synthetic sequence for expression ovalbumin fused to half of a fluorescent protein (BiFC) in plant cells.
gtgccaattcgcatgcggaggtcaacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggacacgctcgagtataagagctcatttttacaacaattaccaacaacaacaaacaacaaacaacattacaattacatttacaattatcgatacaatggccaagctagttttttccctttgttttctgcttttcagtggctgctgcttcgctatggggagtatcggggcagcaagcatggagttttgtttcgacgtctttaaggaacttaaagtccaccatgctaatgaaaatatattttattgccccatcgcaatcatgtctgctttggccatggtttatctgggggccaaggattccaccagaacccagatcaataaagtggtcaggttcgacaaactgcctggattcggggatagcatagaagctcagtgtggcacatcagtgaatgttcattcttctttgagggacatcctgaaccaaatcacaaaacccaacgatgtgtatagtttttcactggctagtcgtctttacgctgaggagaggtacccaattttgcccgagtacctgcaatgtgtaaaagagttgtacagaggaggtttggaacctataaacttccaaaccgcagcagatcaggctcgcgaactcatcaacagttgggttgagagtcaaacaaatggtattatcaggaatgtactccagccatcaagcgtggactcacaaaccgctatggtcctcgtaaatgcaattgtattcaaagggttgtgggagaaggcttttaaagacgaggatacacaggctatgcctttccgcgttactgaacaagaatccaaaccagtacaaatgatgtatcaaatcggactctttcgcgtcgccagcatggcctcagaaaagatgaagatcctcgagctgccattcgcatcagggactatgagcatgctcgtactcttgccagacgaagtttcaggattggaacagcttgagagtataataaattttgagaagctgactgagtggaccagttctaacgttatggaggaaaggaaaattaaagtttaccttcccagaatgaaaatggaagaaaagtacaatctcaccagtgttcttatggctatggggataacagatgtcttttcctcaagcgccaacttgagcggcatatctagtgctgaatccttgaagattagtcaagccgtccatgctgctcatgcagagatcaatgaggcaggccgtgaagttgtgggcagtgctgaagctggggtagatgcagccagtgtctctgaggagttccgtgccgaccatcctttcttgttctgcatcaagcatatcgccacaaacgcagtactcttcttcggtaggtgcgtgagtccttcgggtggtggaggaagtggtggaggaggtagtatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgagtaggcttgtcctgctttaatgagatatgcgagaagcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggaccgctatgggacacgaagtgatccgtttaaactatcagtgtttgacaggatatattggcgggtaaacctaagagaaaagagcgtttattagaataatcggatatttaaaagggcgtgaaaaggtttatccgttcgtccatttgtatgtgccagccgcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggccaattcccgagtgcgcggaacccctatttgtttatttttctaaatacattcaaatatgtatccgctcatgagacaataaccctgataaatgcttcaataatattgaaaaaggaagagtatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaggacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgctcagggaggatatcggggaagaacagtatgtcgagctattttttgacttactggggatcaagcctgattgggagaaaataaaatattatattttactggatgaattgttttagctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggcagatcctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtcttgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagcccctgctcggatctgttggaccggacagtagtcatggttgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggttgaacactct

SEQ ID NO: 36 - DNA, 6418 bp, synthetic construct
  source 1..6418: mol_type = other DNA; note = Synthetic sequence for expressing ovalbumin fused to half of a fluorescent protein (BiFC) in plant cells.
gtgccaattcgcatgcggaggtcaacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggacacgctcgagtataagagctcatttttacaacaattaccaacaacaacaaacaacaaacaacattacaattacatttacaattatcgatacaatggccaagctagttttttccctttgttttctgcttttcagtggctgctgcttcgctgccgaagttgattgttcaagattccccaatgccacagataaagaaggcaaagacgttttggtctgcaacaaggaccttcgccctatttgcggcactgacggggtcacatataccaatgactgtctgttgtgcgcctactccatagaatttgggactaacatatccaaggagcacgatggggaatgcaaagagacagtccccatgaattgctcctcctatgcaaataccactagtgaagatggcaaggtaatggtactctgtaacagggctttcaatccagtgtgtggcactgatggtgttacctatgacaatgaatgtctcttgtgcgcacataaggtggaacaaggtgcctccgtagacaagcgccacgacggaggttgcagaaaggaactcgccgctgtttcagtggattgctccgagtaccctaagcccgattgcacagcagaagatcgtccattgtgtggaagcgataacaagacttatggcaataagtgcaatttctgtaacgccgtagtggagtcaaatgggacccttaccctcagtcactttgggaaatgttcgggtggtggaggaagtggtggaggaggtagtgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaaactgagcaaagaccccaacgagaagcgcgatcacatggtcctgctggagttcgtgaccgccgccgggatcactctcggcatggacgagctgtacaagtaagcttgtcctgctttaatgagatatgcgagaagcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggaccgctatgggacacgaagtgatccgtttaaactatcagtgtttgacaggatatattggcgggtaaacctaagagaaaagagcgtttattagaataatcggatatttaaaagggcgtgaaaaggtttatccgttcgtccatttgtatgtgccagccgcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggccaattcccgagtgcgcggaacccctatttgtttatttttctaaatacattcaaatatgtatccgctcatgagacaataaccctgataaatgcttcaataatattgaaaaaggaagagtatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaggacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgctcagggaggatatcggggaagaacagtatgtcgagctattttttgacttactggggatcaagcctgattgggagaaaataaaatattatattttactggatgaattgttttagctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggcagatcctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtcttgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagcccctgctcggatctgttggaccggacagtagtcatggttgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggttgaacactct

SEQ ID NO: 37 - protein, 4 aa, synthetic construct
HDEL

SEQ ID NO: 38 - protein, 4 aa, synthetic construct
KDEL

SEQ ID NO: 39 - protein, 4 aa, synthetic construct
GGGG

SEQ ID NO: 40 - protein, 4 aa, synthetic construct
KKKK

SEQ ID NO: 41 - protein, 10 aa, synthetic construct
PLSSILRAFY

SEQ ID NO: 42 - protein, 15 aa, synthetic construct
VFAEAIAANSTLVAE

SEQ ID NO: 43 - protein, 8 aa, synthetic construct
DYKDDDDK

SEQ ID NO: 44 - protein, 6 aa, synthetic construct
HHHHHH

SEQ ID NO: 45 - protein, 10 aa, synthetic construct
EQKLISEEDL

SEQ ID NO: 46 - protein, 8 aa, synthetic construct
WSHPQFEK

SEQ ID NO: 47 - protein, 6 aa, synthetic construct
CCPGCC

SEQ ID NO: 48 - protein, 5 aa, synthetic construct
DDDDK

SEQ ID NO: 49 - DNA, 28 bp, synthetic construct
  source 1..28: mol_type = other DNA
atattgtggtgtaaacaaattgacgctt

SEQ ID NO: 50 - DNA, 30 bp, synthetic construct
  source 1..30: mol_type = other DNA
taataaacgctcttttctcttaggtttacc

SEQ ID NO: 51 - protein, 9 aa, synthetic construct
YPYDVPDYA

(19) United States
(12) Patent Application Publication
(10) Pub. No.: US 2024/0049754 A1
(43) Pub. Date: Feb. 15, 2024

(54) COMPOSITIONS, METHODS AND SYSTEMS FOR PRODUCTION OF EGG WHITE PROTEINS IN PLANTS

(71) Applicant: MOZZA FOODS INC., LOS ANGELES, CA (US)

(72) Inventors: CORY J. TOBIN, PASADENA, CA (US); MARSCHAL BELLINGER, LOS ANGELES, CA (US)

(73) Assignee: MOZZA FOODS INC., LOS ANGELES, CA (US)

(21) Appl. No.: 18/202,098

(22) Filed: May 25, 2023

Related U.S. Application Data
(63) Continuation of application No. PCT/US23/65802, filed on Apr. 14, 2023.
(60) Provisional application No. 63/331,460, filed on Apr. 15, 2022.

(51) Int. Cl. A23L 15/00; A23J 3/14; C12N 15/82; C07K 14/77; C07K 14/81

(52) U.S. Cl. CPC A23L 15/35; A23J 3/14; C12N 15/82; C07K 14/77; C07K 14/8135

(57) ABSTRACT

Compositions, methods and systems for producing recombinant egg white proteins, as well as transgenic plants and food compositions comprising recombinant egg white proteins, including tagged proteins comprising egg white proteins with additional amino acids. Egg white proteins can be mixed with plant proteins to produce food products.

### CROSS REFERENCE TO RELATED APPLICATIONS

This application is a Continuation of International Application No. PCT/US23/65802, filed Apr. 14, 2023, which claims the benefit of U.S. Provisional Application No. 63/331,460, filed on Apr. 15, 2022, all of which are incorporated herein by reference in their entirety.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SEQUENCE LISTING

The instant application contains a Sequence Listing which has been submitted electronically in XML format and is hereby incorporated by reference in its entirety. Said XML copy, created on Apr. 10, 2023, is named 62126-702_601_SL.xml and is 97,171 bytes in size.

### BACKGROUND

Chicken (Gallus gallus) eggs are a ubiquitous food for people around the world. Egg whites are a rich source of proteins and carbohydrates that do not contain the low-density lipoprotein associated with egg yolks. Egg white proteins are of particular interest to the scientific and medical communities for their nutritive and antimicrobial properties, but are economically impractical to separate from one another, at large-scale, by conventional chemical and mechanical means.

U.S. patent application Ser. No. 16/701,022 to Anchel teaches recombinant animal-free food compositions and methods of making them. However, Anchel does not teach making food compositions using egg-white proteins and plant proteins.

### SUMMARY

Some aspects of the present disclosure provide an expression cassette for the production of at least one egg white protein in a plant, including: a target sequence capable of encoding an egg white protein; a promoter operatively linked to the target sequence; a translational enhancer operatively linked to the target sequence; a terminator sequence operatively linked to the target sequence; and, optionally, a polynucleotide sequence for localizing or retaining the egg white protein in a destination in a plant cell; and, optionally, a label sequence or a tag that may add additional amino acids that are not part of the native egg white protein and that may remain after post-translational processing in the plant cell.

In some instances, the egg white protein expressed in a plant cell has at least 25%, 30%, 40%, 50%, 60%, 70%, 80%, 85%, 90%, 95%, or 99% sequence identity to the sequence encoding Gallus gallus ovalbumin [SEQ ID NO:5] or the sequence encoding Gallus gallus ovomucoid [SEQ ID NO:6].

Sequence identity can be determined by any suitable method, algorithm, or alignment program, for example, the Smith-Waterman homology search algorithm, the ClustalW, etc. In some instances, sequence identity is determined using an affine gap search with a gap open penalty of 12 and a gap extension penalty of 2, and a BLOSUM matrix of 62.

In some aspects, the expression cassette for producing an egg white protein further comprises a polynucleotide sequence for localizing or retaining the egg white protein in a destination in a plant cell.

In some instances, the expression cassette for producing an egg white protein further comprises a localization sequence encoding a signal peptide for localizing the egg white protein to an endoplasmic reticulum in the plant cell. In some cases, localization sequence is located at the N-terminal of the egg white protein. In some instances, the localization sequence has at least 25%, 30%, 40%, 50%, 60%, 70%, 80%, 85%, 90%, 95%, or 99% sequence identity to SEQ ID NO:7, SEQ ID NO:8, or SEQ ID NO:9.

In some aspects, the expression cassette for producing an egg white protein further comprises a retention sequence for retaining the egg white protein in an endoplasmic reticulum in the plant cell. In some instances, the peptide comprises amino acid sequence comprises HDEL (SEQ ID NO: 37) or KDEL (SEQ ID NO: 38).

In some instances, the expression cassette for producing an egg white protein further comprises a label sequence or a tag that may add additional amino acids to the egg white protein and that may remain after post-translational processing in a plant cell.

In some instances, the expression cassette for producing an egg white protein further comprises a target sequence encoding an egg white protein typically found in avian eggs selected from the group consisting of a chicken, a duck, a turkey, a goose, a guinea hen, a gull, a quail, a pheasant, an ostrich, or an emu. In some instances, the target sequence encoding Gallus gallus ovalbumin comprising at least 25%, 30%, 40%, 50%, 60%, 70%, 80%, 85%, 90%, 95%, or 99% sequence identity to SEQ ID NO:28 or SEQ ID NO:29. In some instances, the expression cassette comprises an amino acid sequence comprising at least 25%, 30%, 40%, 50%, 60%, 70%, 80%, 85%, 90%, 95%, or 99% sequence identity to SEQ ID NO:30.

In some instances, the expression cassette for producing an egg white protein further comprises an enhancer sequence encoding an amino acid sequence comprising at least 25%, 30%, 40%, 50%, 60%, 70%, 80%, 85%, 90%, 95%, or 99% sequence identity to SEQ ID NO:2.

In some aspects, the expression cassette for producing an egg white protein further comprises a localization sequence encoding a signal peptide for localizing the egg white protein in an apoplast of the plant cell. In some instances, the signal peptide comprises an amino acid sequence comprising at least 25%, 30%, 40%, 50%, 60%, 70%, 80%, 85%, 90%, 95%, or 99% sequence identity to SEQ ID NO:7, SEQ ID NO:12, SEQ ID NO:13 or SEQ ID NO:14.

In some aspects, the expression cassette for producing an egg white protein further comprises a localization sequence encoding a signal peptide for localizing the egg white protein in a vacuole of the plant cell. In some instances, the signal peptide comprises an amino acid sequence comprising at least 25%, 30%, 40%, 50%, 60%, 70%, 80%, 85%, 90%, 95%, or 99% sequence identity to SEQ ID NO:7, SEQ ID NO:15, SEQ ID NO:16 or SEQ ID NO:17.

In some aspects, the expression cassette for producing an egg white protein further comprises a localization sequence encoding a signal peptide for localizing the egg white protein in a nucleus of the plant cell. In some instances, the signal peptide comprises an amino acid sequence comprising at least 25%, 30%, 40%, 50%, 60%, 70%, 80%, 85%, 90%, 95%, or 99% sequence identity to SEQ ID NO:18.

In some aspects, the expression cassette further comprises co-expressing a different egg white protein in the plant cell. For example, the expression cassette encodes a first egg white protein that has at least 25%, 30%, 40%, 50%, 60%, 70%, 80%, 85%, 90%, 95%, or 99% sequence identity to Gallus gallus ovalbumin [SEQ ID NO:5], and a second egg white protein that has at least 25%, 30%, 40%, 50%, 60%, 70%, 80%, 85%, 90%, 95%, or 99% sequence identity to Gallus gallus ovomucoid [SEQ ID NO:6].

In some instances, the egg white protein is a tagged protein comprising at least 25%, 30%, 40%, 50%, 60%, 70%, 80%, 85%, 90%, 95%, or 99% homology to the recombinant Gallus gallus ovalbumin produced in a plant transformed with pMOZ183 [SEQ ID NO:32] or pMOZ243 [SEQ ID NO:35], or to the recombinant Gallus gallus ovomucoid produced in a plant transformed with pMOZ184 [SEQ ID NO:33], pMOZ246 [SEQ ID NO:34], or pMOZ244 [SEQ ID NO:36].

Some aspects of the present disclosure provide a plant stably transformed with the expression cassette. Contemplated plants can be a dicot plant, for example, Arabidopsis, tobacco, tomato, potato, sweet potato, cassava, alfalfa, lima bean, pea, chick pea, soybean, carrot, strawberry, lettuce, oak, maple, walnut, rose, mint, squash, daisy, quinoa, buckwheat, mung bean, cow pea, lentil, lupin, peanut, fava bean, French beans, mustard, or cactus. Contemplated plants can also be a monocot plant, for example, turf grass, corn, rice, oat, wheat, barley, sorghum, orchid, iris, lily, onion, palm, or duckweed. In some instances, at least 50% of the leaf epidermal cells in the plant are transfected with the expression cassette and express at least one egg white protein. In some instances, at least 60% of the leaf epidermal cells in the plant are transfected with the expression cassette and express at least one egg white protein. In some instances, at least 70% of the leaf epidermal cells in the plant are transfected with the expression cassette and express at least one egg white protein. In some instances, at least 80% of the leaf epidermal cells in the plant are transfected with the expression cassette and express at least one egg white protein. In some instances, at least 90% of the leaf epidermal cells in the plant are transfected with the expression cassette and express at least one egg white protein. In some instances, at least 95% of the leaf epidermal cells in the plant are transfected with the expression cassette and express at least one egg white protein. In some instances, 100% of the leaf epidermal cells in the plant are transfected with the expression cassette and express at least one egg white protein. In some cases, the plant is transfected with two expression cassettes. In some cases, the plant co-expresses at least two different egg white proteins. In some cases, the plant co-expresses ovomucoid and ovalbumin. In some cases, the expression ratio of ovalbumin to ovomucoid is below 1, below 0.9, or below 0.8. In some cases, the expression ratio of ovalbumin to ovomucoid is above 0.5, above 0.6, or above 0.7. In some cases, the expression ratio of ovalbumin to ovomucoid is about 0.6, about 0.7, or about 0.8.

Some aspects of the present disclosure provide methods for stably expressing an egg white protein in a plant, including: transforming a plant with an expression plasmid including the expression cassette; and growing the transformed plant under conditions wherein the egg white protein is expressed.

Some aspects of the present disclosure provide a food composition including an egg white protein produced using the methods described herein, including, for example, an egg and a processed egg product, an egg-substitute, a frozen dessert, a dessert, a baked good, a topping, an icing, a filling, a low-fat spread, a protein powder, a protein bar, an egg-based dry mix, a soup, a sauce, a salad dressing, a geriatric nutrition, an analogue egg product, a baby formula, an infant formula, a smoothie, a butter alternative, a medical and clinical nutrition product, a sports beverage, a confection, a meat product, a meat analogue, a meal replacement beverage, a weight management food and beverage, protein powder, an ice cream, a mayonnaise, a meringue and meringue powder, or a pancake mix.

Some aspects of the present disclosure provide food composition comprising a recombinant egg white protein, and a plant by-product. In some instances, the recombinant egg white protein has at least 80% sequence identity to the sequence encoding Gallus gallus ovalbumin [SEQ ID NO:5] or sequence encoding Gallus gallus ovomucoid [SEQ ID NO:6].

In some instances, the egg white protein comprises at least one of ovalbumin, ovotransferrin, ovomucoid, ovoglobulin g2, ovoglobulin g3, ovomucin, lysozyme, ovoinhibitor, ovoglycoprotein, flavoprotein, ovomacroglobulin, or any combination thereof. In some instances, the plant by-product comprises a plant protein, a plant lipid, a plant sugar, a plant polyphenol, or plant DNA molecule. In some instances, the plant lipoid comprises at least one of stigmasterol, sitosterol, campesterol, or brassicasterol. In some instances, the plant polyphenol comprises isoflavone. In some instances, the plant DNA comprises plant DNA fragments. In some instances, the plant by-product is derived from a dicot selected from the group consisting of an Arabidopsis, a tobacco, a tomato, a potato, a sweet potato, a cassava, an alfalfa, a lima bean, a pea, a chick pea, a soybean, a carrot, a strawberry, a lettuce, an oak, a maple, a walnut, a rose, a mint, a squash, a daisy, a quinoa, a buckwheat, a mung bean, a cow pea, a lentil, a lupin, a peanut, a fava bean, a French bean, a mustard, or a cactus. In some instances, the plant by-product is derived from a monocot selected from the group consisting of turf grass, corn, rice, oat, wheat, barley, sorghum, orchid, iris, lily, onion, palm, or duckweed.

Some aspects of the present disclosure provide a food composition comprising an egg white protein, wherein the food composition does not comprise a detectable amount of an egg yolk component. As used herein, an egg yolk component refers to a composition typically found in egg yolk but not in egg white. For example, the egg yolk component can be a protein typically found in egg yolk but not in egg white (e.g., vitellin, phosvitin, or lipovitellin). As another example, the egg yolk component can be a phospholipid typically found in egg yolk but not in egg white. In some cases, the egg white protein is a recombinant protein. In some cases, the egg yolk component is an egg yolk protein (e.g., vitellin, phosvitin, or lipovitellin). In some cases, the egg yolk component is a phospholipid.

In some cases, the detectable amount is at or above a concentration of 10⁻⁶ mol/L. In some cases, the detectable amount is at or above a concentration of 10⁻⁷ mol/L. In some cases, the detectable amount is at or above a concentration of 10⁻⁸ mol/L. In some cases, the detectable amount is at or above a concentration of 10⁻⁹ mol/L. In some cases, the detectable amount is at or above a concentration of 10⁻¹⁰ mol/L.

Some aspects of the present disclosure provide a food composition comprising a food composition comprising a recombinant egg white protein, and a plant protein. In some instances, the plant protein is less than 5% (w/w) of total protein content in the composition, less than 3% (w/w) of total protein content in the composition, less than 1% (w/w) of total protein content in the composition, less than 0.5% (w/w) of total protein content in the composition, less than 0.1% (w/w) of total protein content in the composition, less than 0.05% (w/w) of total protein content in the composition, less than 0.01% (w/w) of total protein content in the composition, less than 0.005% (w/w) of total protein content in the composition, less than 0.0001% (w/w) of total protein content in the composition, or less than 0.00005% (w/w) of total protein content in the composition. In some instances, the plant protein is more than 0% (w/w) of total protein content in the composition, more than 0.005% (w/w) of total protein content in the composition, more than 0.01% (w/w) of total protein content in the composition, more than 0.05% (w/w) of total protein content in the composition, more than 0.1% (w/w) of total protein content in the composition, more than 0.5% (w/w) of total protein content in the composition, more than 1% (w/w) of total protein content in the composition, or more than 3% (w/w) of total protein content in the composition. In some instances, plant protein is more than 5% (w/w) of total protein content in the composition, more than 10% (w/w) of total protein content in the composition, more than 20% (w/w) of total protein content in the composition, more than 30% (w/w) of total protein content in the composition, more than 40% (w/w) of total protein content in the composition, more than 50% (w/w) of total protein content in the composition, more than 60% (w/w) of total protein content in the composition, more than 70% (w/w) of total protein content in the composition, more than 80% (w/w) of total protein content in the composition, more than 90% (w/w) of total protein content in the composition, or more than 95% (w/w) of total protein content in the composition.

In some instances, the recombinant egg white protein comprises a plant-specific post-translational modification pattern. In some cases, the post-translational modification is glycosylation, phosphorylation, or sulfation (e.g., tyrosine sulfation). In some cases, the recombinant egg white protein is non-glycosylated, under-glycosylated, or differentially glycosylated. In some cases, the recombinant egg white protein is non-phosphorylated or under-phosphorylated. In some cases, the recombinant egg white protein is non-sulfated or under-sulfated. In some cases, the plant-specific post-translational modification pattern can affect the recombinant egg white protein's properties including stability, solubility, and function. In some cases, the recombinant egg white protein comprises a glycosylation, phosphorylation, or sulfation pattern different from that of a native egg white protein. In some instances, the recombinant egg white protein made in a plant has a glycosylation phosphorylation, or sulfation pattern characteristic of a plant protein. In some instances, the recombinant egg white protein made in a plant has a non-natural glycosylation, phosphorylation, or sulfation pattern. In some instances, the recombinant egg white protein made in a plant has a glycosylation phosphorylation, or sulfation pattern different from that of a native egg white protein.

In some cases, the recombinant egg white protein comprises at least one of ovalbumin, ovotransferrin, ovomucoid, ovoglobulin g2, ovoglobulin g3, ovomucin, lysozyme, ovoinhibitor, ovoglycoprotein, flavoprotein, ovomacroglobulin, or any combination thereof. In some cases, the recombinant egg white protein comprises at least one of ovalbumin, ovotransferrin, ovomucoid, ovoglobulin g2, ovoglobulin g3, ovomucin, lysozyme, ovoinhibitor, ovoglycoprotein, flavoprotein, or ovomacroglobulin. In some cases, the recombinant egg white protein comprises at least three of ovalbumin, ovotransferrin, ovomucoid, ovoglobulin g2, ovoglobulin g3, ovomucin, lysozyme, ovoinhibitor, ovoglycoprotein, flavoprotein, ovomacroglobulin, or any combination thereof. In some cases, the recombinant egg white protein comprises at least four of ovalbumin, ovotransferrin, ovomucoid, ovoglobulin g2, ovoglobulin g3, ovomucin, lysozyme, ovoinhibitor, ovoglycoprotein, flavoprotein, or ovomacroglobulin. In some cases, the recombinant egg white protein comprises at least five of ovalbumin, ovotransferrin, ovomucoid, ovoglobulin g2, ovoglobulin g3, ovomucin, lysozyme, ovoinhibitor, ovoglycoprotein, flavoprotein, or ovomacroglobulin. In some cases, the recombinant egg white protein comprises at least six of ovalbumin, ovotransferrin, ovomucoid, ovoglobulin g2, ovoglobulin g3, ovomucin, lysozyme, ovoinhibitor, ovoglycoprotein, flavoprotein, or ovomacroglobulin.

In some cases, the recombinant egg white protein comprises at most one of ovalbumin, ovotransferrin, ovomucoid, ovoglobulin g2, ovoglobulin g3, ovomucin, lysozyme, ovoinhibitor, ovoglycoprotein, flavoprotein, ovomacroglobulin, or any combination thereof. In some cases, the recombinant egg white protein comprises at most one of ovalbumin, ovotransferrin, ovomucoid, ovoglobulin g2, ovoglobulin g3, ovomucin, lysozyme, ovoinhibitor, ovoglycoprotein, flavoprotein, or ovomacroglobulin. In some cases, the recombinant egg white protein comprises at most three of ovalbumin, ovotransferrin, ovomucoid, ovoglobulin g2, ovoglobulin g3, ovomucin, lysozyme, ovoinhibitor, ovoglycoprotein, flavoprotein, ovomacroglobulin, or any combination thereof. In some cases, the recombinant egg white protein comprises at most four of ovalbumin, ovotransferrin, ovomucoid, ovoglobulin g2, ovoglobulin g3, ovomucin, lysozyme, ovoinhibitor, ovoglycoprotein, flavoprotein, or ovomacroglobulin. In some cases, the recombinant egg white protein comprises at most five of ovalbumin, ovotransferrin, ovomucoid, ovoglobulin g2, ovoglobulin g3, ovomucin, lysozyme, ovoinhibitor, ovoglycoprotein, flavoprotein, or ovomacroglobulin. In some cases, the recombinant egg white protein comprises at most six of ovalbumin, ovotransferrin, ovomucoid, ovoglobulin g2, ovoglobulin g3, ovomucin, lysozyme, ovoinhibitor, ovoglycoprotein, flavoprotein, or ovomacroglobulin.

In some cases, the recombinant egg white protein does not comprise at least one of ovalbumin, ovotransferrin, ovomucoid, ovoglobulin g2, ovoglobulin g3, ovomucin, lysozyme, ovoinhibitor, ovoglycoprotein, flavoprotein, or ovomacroglobulin. In some cases, the recombinant egg white protein does not comprise at least two of ovalbumin, ovotransferrin, ovomucoid, ovoglobulin g2, ovoglobulin g3, ovomucin, lysozyme, ovoinhibitor, ovoglycoprotein, flavoprotein, or ovomacroglobulin.

In some cases, the food composition produced by the methods disclosed herein comprises a plant protein from the plant. In some instances, the plant protein is genetically modified. In some instances, the plant protein is a native plant protein (e.g., not genetically modified). In some instances, the plant protein is a protein from at least one of Arabidopsis, tobacco, tomato, potato, sweet potato, cassava, alfalfa, lima bean, pea, chick pea, soybean, carrot, strawberry, lettuce, oak, maple, walnut, rose, mint, squash, daisy, quinoa, buckwheat, mung bean, cow pea, lentil, lupin, peanut, fava bean, French beans, mustard, cactus, turf grass, corn, rice, oat, wheat, barley, sorghum, orchid, iris, lily, onion, palm, or duckweed. In some cases, the plant protein is a soy protein. In some cases, the plant protein is a soy plant-specific protein. In some cases, the plant protein is a seed storage protein, for example, legumin, vicilin, prolamin, gliadin, β-conglycinin, or glycinin.

In some instances, the recombinant egg white protein is produced in a plant. In some instances, the recombinant egg white protein is made in the same plant where the plant protein is made. In some instances, the recombinant egg white protein is made in the same plant species but in a different plant where the plant protein is made. In some instances, the recombinant egg white protein is made in the different plant species from the plant where the plant protein is made.

In some cases, the food composition is a solid or semi-solid. In some instances, the food composition is homogeneous. In some instances, the food composition is substantially homogeneous. In some cases, the food composition resembles an animal-derived egg white in at least one of color, browning, viscosity, density, rise/height, dome, spread, shape, hardness/firmness, adhesiveness, resilience/recoverable energy, structural integrity/cohesiveness, elasticity/springiness/rebound, chewiness/breakdown, taste, flavor, mouthfeel, or eating quality.

Some aspects of the current disclosure provide a method of producing a food composition, comprising, growing a plant that expresses one or more recombinant egg white proteins; harvesting a portion of the plant; and isolating the recombinant egg white protein from the plant portion. In some instances, the disclosed method further comprises heating the food composition to cause the food composition to solidify. In some cases, the disclosed method further comprises admixing the composition prior to heating the composition. In some instances, heating the composition is performed at a temperature between about 40° C. and about 200° C. In some instances, heating the composition is performed at about 40° C., about 45° C., about 50° C., about 55° C., about 60° C., about 65° C., about 70° C., about 75° C., about 80° C., about 85° C., about 90° C., about 95° C., about 100° C., about 105° C., about 110° C., about 115° C., about 120° C., about 125° C., about 130° C., about 135° C., about 140° C., about 145° C., about 150° C., about 155° C., about 160° C., about 165° C., about 170° C., about 175° C., about 180° C., about 185° C., about 190° C., or about 200° C. In some instances, heating the composition is performed at a range between any of the temperatures described above.

In some instances, heating the composition is performed at a pressure between about atmospheric and about 220 psi. In some instances, the heating the composition is conducted at about 14 psi, about 15 psi, about 16 psi, about 17 psi, about 18 psi, about 19 psi, about 20 psi, about 30 psi, about 40 psi, about 50 psi, about 60 psi, about 70 psi, about 80 psi, about 90 psi, about 100 psi, about 110 psi, about 120 psi, about 130 psi, about 140 psi, about 150 psi, about 160 psi, about 170 psi, about 180 psi, about 190 psi, about 200 psi, about 210 psi, or about 220 psi. In some instances, heating the composition is performed at a range between any of the pressures described above.

In some instances, the disclosed methods produce a food composition in which the plant protein is less than 5% (w/w) of total protein content in the composition, less than 3% (w/w) of total protein content in the composition, less than 1% (w/w) of total protein content in the composition, less than 0.5% (w/w) of total protein content in the composition, less than 0.1% (w/w) of total protein content in the composition, less than 0.05% (w/w) of total protein content in the composition, less than 0.01% (w/w) of total protein content in the composition, less than 0.005% (w/w) of total protein content in the composition, less than 0.0001% (w/w) of total protein content in the composition, or less than 0.00005% (w/w) of total protein content in the composition.

In some instances, the disclosed methods produce a food composition in which the plant protein is more than 0% (w/w) of total protein content in the composition, more than 0.005% (w/w) of total protein content in the composition, more than 0.01% (w/w) of total protein content in the composition, more than 0.05% (w/w) of total protein content in the composition, more than 0.1% (w/w) of total protein content in the composition, more than 0.5% (w/w) of total protein content in the composition, more than 1% (w/w) of total protein content in the composition, or more than 3% (w/w) of total protein content in the composition.

In some instances, the disclosed methods produce a food composition in which the plant protein is more than 5% (w/w) of total protein content in the composition, more than 10% (w/w) of total protein content in the composition, more than 20% (w/w) of total protein content in the composition, more than 30% (w/w) of total protein content in the composition, more than 40% (w/w) of total protein content in the composition, more than 50% (w/w) of total protein content in the composition, more than 60% (w/w) of total protein content in the composition, more than 70% (w/w) of total protein content in the composition, more than 80% (w/w) of total protein content in the composition, more than 90% (w/w) of total protein content in the composition, or more than 95% (w/w) of total protein content in the composition.

In some instances, the disclosed methods produce a food composition that comprises recombinant egg white protein comprising at least one of ovalbumin, ovotransferrin, ovomucoid, ovoglobulin g2, ovoglobulin g3, ovomucin, lysozyme, ovoinhibitor, ovoglycoprotein, flavoprotein, ovomacroglobulin, or any combination thereof. In some instances, the disclosed methods produce a food composition that comprises recombinant egg white protein does not comprise at least one of ovalbumin, ovotransferrin, ovomucoid, ovoglobulin g2, ovoglobulin g3, ovomucin, lysozyme, ovoinhibitor, ovoglycoprotein, flavoprotein, or ovomacroglobulin.

In some instances, the disclosed methods produce a food composition that comprises at least two recombinant egg white proteins selected from the group consisting of ovalbumin, ovotransferrin, ovomucoid, ovoglobulin g2, ovoglobulin g3, ovomucin, lysozyme, ovoinhibitor, ovoglycoprotein, flavoprotein, or ovomacroglobulin. In some instances, the disclosed methods produce a food composition that comprises at least three recombinant egg white proteins selected from the group consisting of ovalbumin, ovotransferrin, ovomucoid, ovoglobulin g2, ovoglobulin g3, ovomucin, lysozyme, ovoinhibitor, ovoglycoprotein, flavoprotein, or ovomacroglobulin. In some instances, the disclosed methods produce a food composition that comprises at least four recombinant egg white proteins selected from the group consisting of ovalbumin, ovotransferrin, ovomucoid, ovoglobulin g2, ovoglobulin g3, ovomucin, lysozyme, ovoinhibitor, ovoglycoprotein, flavoprotein, or ovomacroglobulin. In some instances, the disclosed methods produce a food composition that comprises at least five recombinant egg white proteins selected from the group consisting of ovalbumin, ovotransferrin, ovomucoid, ovoglobulin g2, ovoglobulin g3, ovomucin, lysozyme, ovoinhibitor, ovoglycoprotein, flavoprotein, or ovomacroglobulin. In some instances, the disclosed methods produce a food composition that comprises at least six recombinant egg white proteins selected from the group consisting of ovalbumin, ovotransferrin, ovomucoid, ovoglobulin g2, ovoglobulin g3, ovomucin, lysozyme, ovoinhibitor, ovoglycoprotein, flavoprotein, or ovomacroglobulin.

In some instances, the disclosed methods produce a food composition that comprises at most two recombinant egg white proteins selected from the group consisting of ovalbumin, ovotransferrin, ovomucoid, ovoglobulin g2, ovoglobulin g3, ovomucin, lysozyme, ovoinhibitor, ovoglycoprotein, flavoprotein, or ovomacroglobulin. In some instances, the disclosed methods produce a food composition that comprises at most three recombinant egg white proteins selected from the group consisting of ovalbumin, ovotransferrin, ovomucoid, ovoglobulin g2, ovoglobulin g3, ovomucin, lysozyme, ovoinhibitor, ovoglycoprotein, flavoprotein, or ovomacroglobulin. In some instances, the disclosed methods produce a food composition that comprises at most four recombinant egg white proteins selected from the group consisting of ovalbumin, ovotransferrin, ovomucoid, ovoglobulin g2, ovoglobulin g3, ovomucin, lysozyme, ovoinhibitor, ovoglycoprotein, flavoprotein, or ovomacroglobulin. In some instances, the disclosed methods produce a food composition that comprises at most five recombinant egg white proteins selected from the group consisting of ovalbumin, ovotransferrin, ovomucoid, ovoglobulin g2, ovoglobulin g3, ovomucin, lysozyme, ovoinhibitor, ovoglycoprotein, flavoprotein, or ovomacroglobulin. In some instances, the disclosed methods produce a food composition that comprises at most six recombinant egg white proteins selected from the group consisting of ovalbumin, ovotransferrin, ovomucoid, ovoglobulin g2, ovoglobulin g3, ovomucin, lysozyme, ovoinhibitor, ovoglycoprotein, flavoprotein, or ovomacroglobulin.

In some instances, the methods disclosed herein produce a food composition that has a soy protein. In some instances, the methods disclosed herein produce a food composition in soybean plant. In some cases, the methods disclosed herein produce a food composition that is a liquid. In some cases, the methods disclosed herein produce a food composition that is a solid or semi-solid. In some cases, the methods disclosed herein produce a food composition that is homogeneous or substantially homogeneous.

In some cases, the methods disclosed herein produce a food composition that resembles an animal-derived egg white in at least one of color, browning, viscosity, density, rise/height, dome, spread, shape, hardness/firmness, adhesiveness, resilience/recoverable energy, structural integrity/cohesiveness, elasticity/springiness/rebound, chewiness/breakdown, taste, flavor, mouth-feel, or eating quality.

In some cases, the methods disclosed herein produce a food composition in a plant that is at least one of Arabidopsis, tobacco, tomato, potato, sweet potato, cassava, alfalfa, lima bean, pea, chick pea, soybean, carrot, strawberry, lettuce, oak, maple, walnut, rose, mint, squash, daisy, quinoa, buckwheat, mung bean, cow pea, lentil, lupin, peanut, fava bean, French beans, mustard, cactus, turf grass, corn, rice, oat, wheat, barley, sorghum, orchid, iris, lily, onion, palm, or duckweed. In some instances, the disclosed methods harvest a plant portion that is a seed, leaf, stem, root, flower, or fruit of the plant. In some cases, the methods disclosed herein further comprise crushing the plant portion of the plant. In some cases, the methods disclosed herein further comprises isolating the one or more recombinant egg white proteins from the crushed plant portion. In some instances, isolating the one or more recombinant egg white proteins comprises filtering the crushed plant portion. In some cases, filtering the composition comprises using a microfiltration (MF) membrane to filter the composition. In some cases, filtering the composition comprises using a benchtop tangential flow filtration system to filter the composition. In some cases, the microfiltration (MF) membrane has a pore size between 0.1 and 10 μm. In some cases, the microfiltration (MF) membrane has a pore size between 0.1 and 1 μm. In some cases, microfiltration (MF) membrane has a pore size between 1 and 10 μm. In some cases, the one or more egg white proteins is heated to form a solid or semi-solid.

Certain aspects of the disclosure have other steps or elements in addition to or in place of those mentioned above. The steps or elements will become apparent to those skilled in the art from a reading of the following detailed description when taken with reference to the accompanying drawings.

### BRIEF DESCRIPTION OF THE DRAWINGS

The patent or application file contains at least one drawing executed in color. Copies of this patent or patent application publication with color drawings will be provided by the Office upon request and payment of the necessary fee.

The figures showing embodiments of the system are semi-diagrammatic, and not to scale and, particularly, some of the dimensions are for the clarity of presentation and are shown exaggerated in the figures. Similarly, although the views in the figures for ease of description generally show similar orientations, this depiction in the figures is arbitrary for the most part. Generally, the invention can be operated in any orientation.

FIG. 1 is an example of a map of a pMOZ vector backbone.

FIG. 2 is an example of a map of a pMOZ183 expression plasmid, which is an example expression plasmid for the production of recombinant OVALBUMIN:GFP:FLAG in plants in an embodiment.

FIG. 3A is an image showing expression of recombinant OVALBUMIN:GFP:FLAG from the pMOZ183 expression plasmid in a systemically infected Nicotiana benthamiana plant photographed under ambient white light.

FIG. 3B is an image showing expression of recombinant OVALBUMIN:GFP:FLAG from the pMOZ183 expression plasmid in a systemically infected Nicotiana benthamiana plant photographed under UV light.

FIG. 3C is a magnified image showing expression of recombinant OVALBUMIN:GFP:FLAG from the pMOZ183 expression plasmid in a systemically infected Nicotiana benthamiana plant in leaf epidermal cells photographed at 10× magnification with brightfield.

FIG. 3D is a magnified image showing expression of recombinant OVALBUMIN:GFP:FLAG from the pMOZ183 expression plasmid in a systemically infected Nicotiana benthamiana plant in leaf epidermal cells and with Green Flourescent Protein (GFP) excitation and emission filters.

FIG. 4A1 is a magnified image showing expression of recombinant OVALBUMIN:GFP:FLAG from the pMOZ183 expression plasmid in leaf epidermal cells of a systemically infected Nicotiana benthamiana plant with Red Fluorescent Protein (RFP) excitation and emission filters.

FIG. 4A2 is a magnified image showing expression of recombinant OVALBUMIN:GFP:FLAG from the pMOZ183 expression plasmid in leaf epidermal cells of a systemically infected Nicotiana benthamiana plant with Yellow Fluorescent Protein (YFP) excitation and emission filters.

FIG. 4A3 is a magnified image showing expression of recombinant OVALBUMIN:GFP:FLAG from the pMOZ183 expression plasmid in leaf epidermal cells of a systemically infected Nicotiana benthamiana plant with Green Fluorescent Protein (GFP) excitation and emission filters.

FIG. 4A4 is a magnified image showing expression of recombinant OVALBUMIN:GFP:FLAG from the pMOZ183 expression plasmid in leaf epidermal cells of a systemically infected Nicotiana benthamiana plant with superimposed excitation and emission filters.

FIG. 4B1 is a magnified image showing expression of recombinant OVALBUMIN:GFP:FLAG from the pMOZ183 expression plasmid in leaf epidermal cells of a systemically infected Nicotiana benthamiana plant with Red Fluorescent Protein (RFP) excitation and emission filters.

FIG. 4B2 is a magnified image showing expression of recombinant OVALBUMIN:GFP:FLAG from the pMOZ183 expression plasmid in leaf epidermal cells of a systemically infected Nicotiana benthamiana plant with Yellow Fluorescent Protein (YFP) excitation and emission filters.

FIG. 4B3 is a magnified image showing expression of recombinant OVALBUMIN:GFP:FLAG from the pMOZ183 expression plasmid in leaf epidermal cells of a systemically infected Nicotiana benthamiana plant with Green Fluorescent Protein (GFP) excitation and emission filters.

FIG. 4B4 is a magnified image showing expression of recombinant OVALBUMIN:GFP:FLAG from the pMOZ183 expression plasmid in leaf epidermal cells of a systemically infected Nicotiana benthamiana plant with superimposed excitation and emission filters.

FIG. 4C1 is a magnified image showing expression of recombinant OVALBUMIN:GFP:FLAG from the pMOZ183 expression plasmid in leaf epidermal cells of a systemically infected Nicotiana benthamiana plant with Red Fluorescent Protein (RFP) excitation and emission filters.

FIG. 4C2 is a magnified image showing expression of recombinant OVALBUMIN:GFP:FLAG from the pMOZ183 expression plasmid in leaf epidermal cells of a systemically infected Nicotiana benthamiana plant with Yellow Fluorescent Protein (YFP) excitation and emission filters.

FIG. 4C3 is a magnified image showing expression of recombinant OVALBUMIN:GFP:FLAG from the pMOZ183 expression plasmid in leaf epidermal cells of a systemically infected Nicotiana benthamiana plant with Green Fluorescent Protein (GFP) excitation and emission filters.

FIG. 4C4 is a magnified image showing expression of recombinant OVALBUMIN:GFP:FLAG from the pMOZ183 expression plasmid in leaf epidermal cells of a systemically infected Nicotiana benthamiana plant with superimposed excitation and emission filters.

FIG. 5A1 is an image showing expression of recombinant OVALBUMIN:GFP:FLAG from the pMOZ183 expression plasmid in Glycine max protoplasts with Red Fluorescent Protein (RFP) excitation and emission filters.

FIG. 5A2 is an image showing expression of recombinant OVALBUMIN:GFP:FLAG from the pMOZ183 expression plasmid in Glycine max protoplasts with Yellow Fluorescent Protein (YFP) excitation and emission filters.

FIG. 5A3 is an image showing expression of recombinant OVALBUMIN:GFP:FLAG from the pMOZ183 expression plasmid in Glycine max protoplasts with Green Fluorescent Protein (GFP) excitation and emission filters.

FIG. 5A4 is an image showing expression of recombinant OVALBUMIN:GFP:FLAG from the pMOZ183 expression plasmid in Glycine max protoplasts with superimposed excitation and emission filters.

FIG. 5B1 is an image showing expression of recombinant OVALBUMIN:GFP:FLAG from the pMOZ183 expression plasmid in Glycine max protoplasts with Red Fluorescent Protein (RFP) excitation and emission filters.

FIG. 5B2 is an image showing expression of recombinant OVALBUMIN:GFP:FLAG from the pMOZ183 expression plasmid in Glycine max protoplasts with Yellow Fluorescent Protein (YFP) excitation and emission filters.

FIG. 5B3 is an image showing expression of recombinant OVALBUMIN:GFP:FLAG from the pMOZ183 expression plasmid in Glycine max protoplasts with Green Fluorescent Protein (GFP) excitation and emission filters.

FIG. 5B4 is an image showing expression of recombinant OVALBUMIN:GFP:FLAG from the pMOZ183 expression plasmid in Glycine max protoplasts with superimposed excitation and emission filters.

FIG. 5C1 is an image showing expression of recombinant OVALBUMIN:GFP:FLAG from the pMOZ183 expression plasmid in Glycine max protoplasts with Red Fluorescent Protein (RFP) excitation and emission filters.

FIG. 5C2 is an image showing expression of recombinant OVALBUMIN:GFP:FLAG from the pMOZ183 expression plasmid in Glycine max protoplasts with Yellow Fluorescent Protein (YFP) excitation and emission filters.

FIG. 5C3 is an image showing expression of recombinant OVALBUMIN:GFP:FLAG from the pMOZ183 expression plasmid in Glycine max protoplasts with Green Fluorescent Protein (GFP) excitation and emission filters.

FIG. 5C4 is an image showing expression of recombinant OVALBUMIN:GFP:FLAG from the pMOZ183 expression plasmid in Glycine max protoplasts with superimposed excitation and emission filters.

FIG. 6 is an example of a map of a pMOZ184 expression plasmid in an embodiment.

FIG. 7A is an image showing expression of recombinant OVOMUCOID:GFP:FLAG from the pMOZ184 expression plasmid in a systemically infected Nicotiana benthamiana plant under ambient white light.

FIG. 7B is an image showing expression of recombinant OVOMUCOID:GFP:FLAG from the pMOZ184 expression plasmid in a systemically infected Nicotiana benthamiana plant under UV light.

FIG. 7C is an image showing expression of recombinant OVOMUCOID:GFP:FLAG from the pMOZ184 expression plasmid in a systemically infected Nicotiana benthamiana plant with brightfield.

FIG. 7D is an image showing expression of recombinant OVOMUCOID:GFP:FLAG from the pMOZ184 expression plasmid in a systemically infected Nicotiana benthamiana plant with Green Fluorescent Protein (GFP) excitation and emission filters.

FIG. 8A1 is an image showing expression of recombinant OVOMUCOID:GFP:FLAG from the pMOZ184 expression plasmid in leaf epidermal cells of a systemically infected Nicotiana benthamiana plant with Red Fluorescent Protein (RFP) excitation and emission filters.

FIG. 8A2 is an image showing expression of recombinant OVOMUCOID:GFP:FLAG from the pMOZ184 expression plasmid in leaf epidermal cells of a systemically infected Nicotiana benthamiana plant with Yellow Fluorescent Protein (YFP) excitation and emission filters.

FIG. 8A3 is an image showing expression of recombinant OVOMUCOID:GFP:FLAG from the pMOZ184 expression plasmid in leaf epidermal cells of a systemically infected Nicotiana benthamiana plant with Green Fluorescent Protein (GFP) excitation and emission filters.

FIG. 8A4 is an image showing expression of recombinant OVOMUCOID:GFP:FLAG from the pMOZ184 expression plasmid in leaf epidermal cells of a systemically infected Nicotiana benthamiana plant with superimposed excitation and emission filters.

FIG. 8B1 is an image showing expression of recombinant OVOMUCOID:GFP:FLAG from the pMOZ184 expression plasmid in leaf epidermal cells of a systemically infected Nicotiana benthamiana plant with Red Fluorescent Protein (RFP) excitation and emission filters.

FIG. 8B2 is an image showing expression of recombinant OVOMUCOID:GFP:FLAG from the pMOZ184 expression plasmid in leaf epidermal cells of a systemically infected Nicotiana benthamiana plant with Yellow Fluorescent Protein (YFP) excitation and emission filters.

FIG. 8B3 is an image showing expression of recombinant OVOMUCOID:GFP:FLAG from the pMOZ184 expression plasmid in leaf epidermal cells of a systemically infected Nicotiana benthamiana plant with Green Fluorescent Protein (GFP) excitation and emission filters.

FIG. 8B4 is an image showing expression of recombinant OVOMUCOID:GFP:FLAG from the pMOZ184 expression plasmid in leaf epidermal cells of a systemically infected Nicotiana benthamiana plant with superimposed excitation and emission filters.

FIG. 8C1 is an image showing expression of recombinant OVOMUCOID:GFP:FLAG from the pMOZ184 expression plasmid in leaf epidermal cells of a systemically infected Nicotiana benthamiana plant with Red Fluorescent Protein (RFP) excitation and emission filters.

FIG. 8C2 is an image showing expression of recombinant OVOMUCOID:GFP:FLAG from the pMOZ184 expression plasmid in leaf epidermal cells of a systemically infected Nicotiana benthamiana plant with Yellow Fluorescent Protein (YFP) excitation and emission filters.

FIG. 8C3 is an image showing expression of recombinant OVOMUCOID:GFP:FLAG from the pMOZ184 expression plasmid in leaf epidermal cells of a systemically infected Nicotiana benthamiana plant with Green Fluorescent Protein (GFP) excitation and emission filters.

FIG. 8C4 is an image showing expression of recombinant OVOMUCOID:GFP:FLAG from the pMOZ184 expression plasmid in leaf epidermal cells of a systemically infected Nicotiana benthamiana plant with superimposed excitation and emission filters.

FIG. 9 is an example of a map of a pMOZ246 expression plasmid, which is an example expression plasmid for the production of recombinant OVOMUCOID:BFP:HA in plants according to the present invention.

FIG. 10A1 is an image showing expression of recombinant OVALBUMIN:GFP:FLAG from the pMOZ183 expression plasmid in leaf epidermal cells of systemically infected Nicotiana benthamiana plants transformed with pMOZ183 alone with Red Fluorescent Protein (RFP) excitation and emission filters.

FIG. 10A2 is an image showing expression of recombinant OVALBUMIN:GFP:FLAG from the pMOZ183 expression plasmid in leaf epidermal cells of systemically infected Nicotiana benthamiana plants transformed with pMOZ183 alone with Green Fluorescent Protein (GFP) excitation and emission filters.

FIG. 10A3 is an image showing expression of recombinant OVALBUMIN:GFP:FLAG from the pMOZ183 expression plasmid in leaf epidermal cells of systemically infected Nicotiana benthamiana plants transformed with pMOZ183 alone with Blue Fluorescent Protein (BFP) excitation and emission filters.

FIG. 10A4 is an image showing expression of recombinant OVALBUMIN:GFP:FLAG from the pMOZ183 expression plasmid in leaf epidermal cells of systemically infected Nicotiana benthamiana plants transformed with pMOZ183 alone with superimposed excitation and emission filters.

FIG. 10B1 is an image showing expression of recombinant OVOMUCOID:BFP:HA from the pMOZ246 expression plasmid in leaf epidermal cells of systemically infected Nicotiana benthamiana plants transformed with pMOZ246 alone with Red Fluorescent Protein (RFP) excitation and emission filters.

FIG. 10B2 is an image showing expression of recombinant OVOMUCOID:BFP:HA from the pMOZ246 expression plasmid in leaf epidermal cells of systemically infected Nicotiana benthamiana plants transformed with pMOZ246 alone with Green Fluorescent Protein (GFP) excitation and emission filters.

FIG. 10B3 is an image showing expression of recombinant OVOMUCOID:BFP:HA from the pMOZ246 expression plasmid in leaf epidermal cells of systemically infected Nicotiana benthamiana plants transformed with pMOZ246 alone with Blue Fluorescent Protein (BFP) excitation and emission filters.

FIG. 10B4 is an image showing expression of recombinant OVOMUCOID:BFP:HA from the pMOZ246 expression plasmid in leaf epidermal cells of systemically infected Nicotiana benthamiana plants transformed with pMOZ246 alone with superimposed excitation and emission filters.

FIG. 10C1 is an image showing expression of recombinant OVALBUMIN:GFP:FLAG from the pMOZ183 expression plasmid and recombinant OVOMUCOID:BFP:HA from the pMOZ246 expression plasmid in leaf epidermal cells of systemically infected Nicotiana benthamiana plants transformed with pMOZ183 and pMOZ246 together with Red Fluorescent Protein (RFP) excitation and emission filters.

FIG. 10C2 is an image showing expression of recombinant OVALBUMIN:GFP:FLAG from the pMOZ183 expression plasmid and recombinant OVOMUCOID:BFP:HA from the pMOZ246 expression plasmid in leaf epidermal cells of systemically infected Nicotiana benthamiana plants transformed with pMOZ183 and pMOZ246 together with Green Fluorescent Protein (GFP) excitation and emission filters.

FIG. 10C3 is an image showing expression of recombinant OVALBUMIN:GFP:FLAG from the pMOZ183 expression plasmid and recombinant OVOMUCOID:BFP:HA from the pMOZ246 expression plasmid in leaf epidermal cells of systemically infected Nicotiana benthamiana plants transformed with pMOZ183 and pMOZ246 together with Blue Fluorescent Protein (BFP) excitation and emission filters.

FIG. 10C4 is an image showing expression of recombinant OVALBUMIN:GFP:FLAG from the pMOZ183 expression plasmid and recombinant OVOMUCOID:BFP:HA from the pMOZ246 expression plasmid in leaf epidermal cells of systemically infected Nicotiana benthamiana plants transformed with pMOZ183 and pMOZ246 together with superimposed excitation and emission filters.

FIG. 11A is a graph showing quantitative analysis of recombinant OVALBUMIN:GFP:FLAG from the pMOZ183 expression plasmid in leaf epidermal cells of systemically infected Nicotiana benthamiana plants transformed with pMOZ183 alone.

FIG. 11B is a graph showing quantitative analysis of recombinant OVOMUCOID:BFP:HA from the pMOZ246 expression plasmid in leaf epidermal cells of systemically infected Nicotiana benthamiana plants transformed with pMOZ246.

FIG. 11C is a graph showing quantitative analysis of recombinant OVALBUMIN:GFP:FLAG from the pMOZ183 expression plasmid and recombinant OVOMUCOID:BFP:HA from the pMOZ246 expression plasmid in leaf epidermal cells of systemically infected Nicotiana benthamiana plants transformed with pMOZ183 and pMOZ246 together.

FIG. 12A is a graph showing Pearson's correlation regression analysis applied to fluorescence intensities measured across lines plotted for the relative fluorescence intensity measured for ovomucoid:BFP; HA versus ovalbumin: GFP: FLAG.

FIG. 12B is a graph showing Pearson's correlation regression analysis applied to fluorescence intensities measured across lines plotted for the relative fluorescence intensity measured for ovomucoid:BFP; HA versus leaf epidermal cell walls.

FIG. 12C is a graph showing Pearson's correlation regression analysis applied to fluorescence intensities measured across lines plotted for the relative fluorescence intensity measured for ovalbumin:GFP:FLAG versus leaf epidermal cell walls.

FIG. 12D is a bar graph showing the average R value for the regression analyses of the lines plotted for BFP and GFP fluorescence intensity (black bar), for the regression analyses of the lines plotted for BFP and RFP fluorescence intensity (dark gray bar), and for the regression analyses of the lines plotted for GFP and RFP fluorescence intensity (light gray bar).

FIG. 13A shows the results from colony PCR of Agrobacterium tumefaciens carrying the pMOZ183 expression plasmid alone.

FIG. 13B shows the results from colony PCR of Agrobacterium tumefaciens carrying the pMOZ246 expression plasmid alone.

FIG. 14A show the results of Western blot analysis showing the detection of recombinant OVALBUMIN:GFP:FLAG from the pMOZ183 expression plasmid and recombinant OVOMUCOID:BFP:HA from the pMOZ246 expression plasmid, in leaf epidermal cells of systemically infected Nicotiana benthamiana plants transformed with pMOZ183 alone, pMOZ246 alone, or pMOZ183 and pMOZ246 together.

FIG. 14B shows the results of SDS-PAGE analysis showing the detection of recombinant OVALBUMIN:GFP:FLAG from the pMOZ183 expression plasmid and recombinant OVOMUCOID:BFP:HA from the pMOZ246 expression plasmid, in leaf epidermal cells of systemically infected Nicotiana benthamiana plants transformed with pMOZ183 alone, pMOZ246 alone, or pMOZ183 and pMOZ246 together.

FIG. 14C show the results of Western blot analysis showing the detection of recombinant OVALBUMIN:GFP:FLAG from the pMOZ183 expression plasmid and recombinant OVOMUCOID:BFP:HA from the pMOZ246 expression plasmid, in leaf epidermal cells of systemically infected Nicotiana benthamiana plants transformed with pMOZ183 alone, pMOZ246 alone, or pMOZ183 and pMOZ246 together,

FIG. 14D shows the results of SDS-PAGE analysis showing the detection of recombinant OVALBUMIN:GFP:FLAG from the pMOZ183 expression plasmid and recombinant OVOMUCOID:BFP:HA from the pMOZ246 expression plasmid, in leaf epidermal cells of systemically infected Nicotiana benthamiana plants transformed with pMOZ183 alone, pMOZ246 alone, or pMOZ183 and pMOZ246 together.

FIG. 14E shows the results of Western Blot analysis showing the detection of recombinant OVALBUMIN:GFP:FLAG from the pMOZ183 expression plasmid and recombinant OVOMUCOID:BFP:HA from the pMOZ246 expression plasmid, in leaf epidermal cells of systemically infected Nicotiana benthamiana plants transformed with pMOZ183 alone, pMOZ246 alone, or pMOZ183 and pMOZ246 together.

FIG. 14F shows the results of SDS-PAGE analysis showing the detection of recombinant OVALBUMIN:GFP:FLAG from the pMOZ183 expression plasmid and recombinant OVOMUCOID:BFP:HA from the pMOZ246 expression plasmid, in leaf epidermal cells of systemically infected Nicotiana benthamiana plants transformed with pMOZ183 alone, pMOZ246 alone, or pMOZ183 and pMOZ246 together.

FIG. 15A is an example of a map of a pMOZ243 expression plasmid, which is an example expression plasmid for the production of recombinant OVALBUMIN:nVENUS in plants according to the present disclosure.

FIG. 15B is an example of a map of a pMOZ243 expression plasmid, which is an example expression plasmid for the production of recombinant OVOMUCOID:cVENUS in plants according to the present disclosure.

FIG. 16A1 is an image showing expression of recombinant OVALBUMIN:nVENUS from the pMOZ243 expression plasmid and recombinant OVOMUCOID:cVENUS from the pMOZ244 expression plasmid in leaf epidermal cells of systemically infected Nicotiana benthamiana plants transformed with pMOZ243 alone.

FIG. 16A2 is an image showing expression of recombinant OVALBUMIN:nVENUS from the pMOZ243 expression plasmid and recombinant OVOMUCOID:cVENUS from the pMOZ244 expression plasmid in leaf epidermal cells of systemically infected Nicotiana benthamiana plants transformed with pMOZ243 alone,

FIG. 16A3 is an image showing expression of recombinant OVALBUMIN:nVENUS from the pMOZ243 expression plasmid and recombinant OVOMUCOID:cVENUS from the pMOZ244 expression plasmid in leaf epidermal cells of systemically infected Nicotiana benthamiana plants transformed with pMOZ243 alone,

FIG. 16B1 is an image showing expression of recombinant OVALBUMIN:nVENUS from the pMOZ243 expression plasmid and recombinant OVOMUCOID:cVENUS from the pMOZ244 expression plasmid in leaf epidermal cells of systemically infected Nicotiana benthamiana plants transformed with pMOZ244 alone.

FIG. 16B2 is an image showing expression of recombinant OVALBUMIN:nVENUS from the pMOZ243 expression plasmid and recombinant OVOMUCOID:cVENUS from the pMOZ244 expression plasmid in leaf epidermal cells of systemically infected Nicotiana benthamiana plants transformed with pMOZ244 alone.

FIG. 16B3 is an image showing expression of recombinant OVALBUMIN:nVENUS from the pMOZ243 expression plasmid and recombinant OVOMUCOID:cVENUS from the pMOZ244 expression plasmid in leaf epidermal cells of systemically infected Nicotiana benthamiana plants transformed with pMOZ244 alone.

FIG. 16C1 is an image showing expression of recombinant OVALBUMIN:nVENUS from the pMOZ243 expression plasmid and recombinant OVOMUCOID:cVENUS from the pMOZ244 expression plasmid in leaf epidermal cells of systemically infected Nicotiana benthamiana plants transformed with pMOZ243 and pMOZ244 together.

FIG. 16C2 is an image showing expression of recombinant OVALBUMIN:nVENUS from the pMOZ243 expression plasmid and recombinant OVOMUCOID:cVENUS from the pMOZ244 expression plasmid in leaf epidermal cells of systemically infected Nicotiana benthamiana plants transformed with pMOZ243 and pMOZ244 together.

FIG. 16C3 is an image showing expression of recombinant OVALBUMIN:nVENUS from the pMOZ243 expression plasmid and recombinant OVOMUCOID:cVENUS from the pMOZ244 expression plasmid in leaf epidermal cells of systemically infected Nicotiana benthamiana plants transformed with pMOZ243 and pMOZ244 together.

FIG. 17 is a flow-chart showing an illustrative process for producing a food composition including a recombinant egg white protein.

### DETAILED DESCRIPTION

The following embodiments are described in sufficient detail to enable those skilled in the art to make and use the invention. It is to be understood that other embodiments would be evident based on the present disclosure, and that system, process, or mechanical changes can be made without departing from the scope of an embodiment of the present disclosure.

In the following description, numerous specific details are given to provide a thorough understanding of the invention. However, it will be apparent that the invention can be practiced without these specific details. In order to avoid obscuring an embodiment of the present disclosure, some well-known techniques, system configurations, and process steps are not disclosed in detail. Throughout this disclosure, various publications, patents and published patent specifications are referenced by an identifying citation. The disclosures of these publications, patents and published patent specifications are hereby incorporated by reference into the present disclosure.

### Definitions

These and other valuable aspects of the embodiments of the present disclosure consequently further the state of the technology to at least the next level. While the disclosure has been described in conjunction with a specific best mode, it is to be understood that many alternatives, modifications, and variations will be apparent to those skilled in the art in light of the descriptions herein. Accordingly, it is intended to embrace all such alternatives, modifications, and variations that fall within the scope of the included claims. All matters set forth herein or shown in the accompanying drawings are to be interpreted in an illustrative and non-limiting sense.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

Any systems, methods, software, and platforms described herein are modular and not limited to sequential steps. Accordingly, terms such as “first” and “second” do not necessarily imply priority, order of importance, or order of acts.

As used herein, the term “about” or the symbol “—” when referring to a number or a numerical range means that the number or numerical range referred to is an approximation within experimental variability (or within statistical experimental error), and the number or numerical range may vary from, for example, from 1% to 10% of the stated number or numerical range. Unless otherwise indicated by context, the term “about” refers to ±10% of a stated number or value.

As used herein, the term “approximately” means within an acceptable error range for the particular value as determined by one of ordinary skill in the art, which will depend in part on how the value is measured or determined, e.g., the limitations of the measurement system. For example, “approximately” can mean within 1 or more than 1 standard deviation, per the practice in the given value. Where particular values are described in the application and claims, unless otherwise stated the term “approximately” should be assumed to mean an acceptable error range for the particular value.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

All ranges disclosed herein also encompass any and all possible sub-ranges and combinations of sub-ranges thereof. Any listed range can be recognized as sufficiently describing and enabling the same range being broken down into at least equal halves, thirds, quarters, fifths, tenths, and so forth. As a non-limiting example, each range discussed herein can be readily broken down into a lower third, middle third and upper third, and the like. All language such as “up to,” “at least,” “greater than,” “less than,” and the like include the number recited and refer to ranges which can be subsequently broken down into sub-ranges as discussed above. Finally, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Whenever the term “at least,” “greater than,” “greater than or equal to”, or a similar phrase precedes the first numerical value in a series of two or more numerical values, the term “at least,” “greater than,” “greater than or equal to” or similar phrase applies to each of the numerical values in that series of numerical values. For example, “at least 1, 2, or 3” is equivalent to “at least 1, at least 2, and/or at least 3.”

Whenever the term “no more than,” “less than,” “less than or equal to,” “no greater than,” “at most” or a similar phrase, precedes the first numerical value in a series of two or more numerical values, the term “no more than,” “less than,” “less than or equal to,” “no greater than,” “at most,” or similar phrase applies to each of the numerical values in that series of numerical values. For example, “less than 3, 2, or 1” is equivalent to “less than 3, less than 2, and/or less than 1.”

As used herein, the following meanings apply unless otherwise specified. The word “may” is used in a permissive sense (i.e., meaning having the potential to), rather than the mandatory sense (i.e., meaning must). The words “include”, “including”, and “includes” and the like mean including, but not limited to. The singular forms “a,” “an,” and “the” include plural referents. Thus, for example, reference to “an element” includes a combination of two or more elements, notwithstanding use of other terms and phrases for one or more elements, such as “one or more.” The phrase “at least one” includes “one”, “one or more”, “one or a plurality” and “a plurality”. The term “or” is, unless indicated otherwise, non-exclusive, i.e., encompassing both “and” and “or.” The term “any of” between a modifier and a sequence means that the modifier modifies each member of the sequence. So, for example, the phrase “at least any of 1, 2 or 3” means “at least 1, at least 2 or at least 3”. The term “consisting essentially of” refers to the inclusion of recited elements and other elements that do not materially affect the basic and novel characteristics of a claimed combination.

Where a definition or use of a term in an incorporated reference is inconsistent or contrary to the definition of that term provided herein, the definition of that term provided herein applies and the definition of that term in the reference does not apply.

As used herein, a “vector” is a plasmid comprising operably linked polynucleotide sequences that facilitate expression of a coding sequence in a particular host organism (e.g., a bacterial expression vector or a plant expression vector). Polynucleotide sequences that facilitate expression in prokaryotes can include, e.g., a promoter, an enhancer, an operator, and a ribosome binding site, often along with other sequences. Eukaryotic cells can use promoters, enhancers, termination and polyadenylation signals and other sequences that are generally different from those used by prokaryotes.

As used herein, an “expression cassette” refers to a nucleic acid construct that includes elements for expressing a target product, such as a recombinant egg white protein. As an example, “expression cassette” refers to a nucleic acid construct including a target sequence encoding the target product, and an expression regulatory region that is operatively linked to the target sequence. The term “encoding” refers to translation and transcription.

As used herein, “operatively linked” in regard to a target sequence means that the target sequence encoding the target product is located at an appropriate position with respect to an expression regulatory region so that the target product can be expressed.

As used herein, the term “differentially-glycosylated” means the absence of glycans on the protein or the glycosylation of the protein with 1 or more N or O linked glycans not commonly found on the protein in the native organism.

As used herein, an “expression regulatory region” refers to a region which controls the expression of the target sequence in an expression cassette. Specifically, as an example, “expression regulatory region” refers to a region including a promoter. The expression regulatory region can further include various regulatory sequences involved in transcriptional and translational regulation. Examples of the promoter can include any sequence that signals initiation of transcription, and as a specific example includes a truncated version of the constitutive Cauliflower Mosaic Virus 35S promoter [SEQ ID NO:1], AtuMas Pro+5′UTR, RbcS2 promoter, a soybean GY1 Promoter, soybean CG1 Promoter, or other suitable promoters.

In the expression regulatory region in the expression cassette as an example, a sequence corresponding to a translational enhancer. As used herein, a “translational enhancer” is a nucleic acid sequence located around a promoter for the purpose of increasing the translation of the target product in plants. Examples of a translational enhancer include any sequence that enhances translation, and as a specific example includes the omega leader sequence of the Tobacco Mosaic Virus [SEQ ID NO:2].

In the expression regulatory region in the expression cassette as an example, a sequence corresponding to a terminator sequence. As used herein, a “terminator sequence” refers to a nucleic acid sequence that signals transcription termination and can increase mRNA stability through polyadenylation. Examples of terminator sequences include any sequence that signals termination of transcription, and as specific examples include sequences encoding nopaline synthase terminator [SEQ ID NO:3], sequences encoding octopine synthase terminator [SEQ ID NO:4], Octopine (OCS) terminator, and NOS terminator that serve as universal termination sequences for plant gene expression and can increase mRNA stability through polyadenylation.

As an example, the expression cassette for a target product can be created by operatively linking a target sequence to an expression regulatory region including a promoter, a translational enhancer, and a termination sequence. As used herein, “target sequence” refers to a nucleic acid sequence encoding the target product. Examples of target sequences include any nucleic acid sequence that encodes an egg white protein, and as specific examples include sequences encoding Gallus gallus ovalbumin [SEQ ID NO:5] and sequences encoding Gallus gallus ovomucoid [SEQ ID NO:6] that have been codon optimized for expression in plants.

As an example, the expression cassette for a target product can optionally include a nucleic acid sequence which controls the localization or retention of the target product in a destination in a plant cell. Examples of such sequences include any sequence that localizes or retains the target product in the endoplasmic reticulum, apoplast, protein storage vacuole or other vacuole, or nucleus of a plant cell. As a further example, the target product may be localized to the cytoplasm by deleting nucleic acid sequences encoding a native signal peptide.

It is contemplated that the peptide that controls the localization of a target product in a destination in a plant cell can be located in either the N-terminal or C-terminal end of the target product. As used herein, a “retention sequence” refers to a nucleic acid sequence encoding a peptide at the N-terminal or the C-terminal end of the target product which controls the retention of the target product in a destination in a plant cell.

Specific examples of N-terminal localization sequences for localizing the target product in the endoplasmic reticulum include sequences encoding soybean GY1 signal peptide [SEQ ID NO:7], sequences encoding Arabidopsis thaliana 2S2 signal peptide [SEQ ID NO:8], and sequences encoding soybean CG1 signal peptide [SEQ ID NO:9]. Specific examples of retention sequences for retaining the target product in the endoplasmic reticulum include sequences encoding the peptide HDEL (SEQ ID NO: 37) (which in some instances can be coded by DNA sequence SEQ ID NO:10]) and sequences encoding the peptide KDEL (SEQ ID NO: 38) which in some instances can be coded by DNA sequence [SEQ ID NO:11].

Specific examples localization sequences encoding a signal peptide located at the N-terminal of the target product for localizing the target product in the apoplast include sequences encoding soybean GY1 signal peptide [SEQ ID NO:7] and sequences encoding soybean GY4 signal peptide [SEQ ID NO:12]. Specific examples of localization sequences for localizing the target product in the apoplast encoding a signal peptide located at the C-terminal of the target product include sequences encoding the peptide GGGG [SEQ ID NO:39] which in some instances can be coded by DNA sequence [SEQ ID NO:13] and sequences encoding the peptide KKKK [SEQ ID NO:40] which in some instances can be coded by DNA sequence [SEQ ID NO:14].

Specific examples localization sequences encoding a signal peptide located at the N-terminal of the target product for localizing the target product in the vacuole include sequences encoding soybean GY1 signal peptide [SEQ ID NO:7] and sequences encoding soybean GY5 signal peptide [SEQ ID NO:15]. Specific examples of localization sequences encoding a signal peptide located at the C-terminal of the target product for localizing the target product in the vacuole include sequences encoding the peptide PLSSILRAFY [SEQ ID NO:41] (which in some instances can be coded by DNA sequence [SEQ ID NO:16]) and sequences encoding the peptide VFAEAIAANSTLVAE [SEQ ID NO:42] (which in some instances can be coded by DNA sequence [SEQ ID NO:17]).

Specific examples of N-terminal localization sequences for localizing the target product in the nucleus include sequences encoding the SV40 nuclear localization signal [SEQ ID NO:18].

The expression cassette for a target product can optionally include a label sequence. As used herein, “label sequence” refers to nucleic acid sequences encoding regions of the target product which can be used to visualize the target product using imaging techniques such as fluorescence microscopy. Examples of label sequences include any nucleic acid sequence that enables visualization of the target product by fluorescence microscopy, and as specific examples include sequences encoding proteins derived from the Aequorea victoria Green Fluorescent Protein gene (avGFP), the Aequorea victoria mox Blue Fluorescent Protein (moxBFP), and other fluorescent proteins that enable detection of the target product by fluorescence microscopy. Linker sequences can optionally be included to fuse the target sequence to the label sequence that enables visualization of the target product using imaging techniques.

As an example, the expression cassette for a target product can optionally include a sequence encoding a tag. In some instances, a tag can be a peptide that enables detection of the target product using protein analysis methods such as SDS-PAGE and Western blot analysis. Contemplated tags include FLAG-tag (DYKDDDDK) (SEQ ID NO: 43), His-tag (HHHHHH) (SEQ ID NO: 44), Myc-tag (EQKLISEEDL) (SEQ ID NO: 45), Strep-tag (WSHPQFEK) (SEQ ID NO: 46), TC tag (CCPGCC) (SEQ ID NO: 47), peptide DDDDK (SEQ ID NO: 48) encoded in [SEQ ID NO:19] and HA-tag (YPYDVPDYA) (SEQ ID NO: 51) (in some cases can be coded by DNA sequence in [SEQ ID NO:20].

As an example, the expression cassette for a target product can optionally include paired sequences to enable co-localization of one or more target products in a plant cell. Examples of paired sequences any nucleic acid sequences that enable co-localization of one or more target products in the plant cell, and as specific examples include sequences encoding the N-terminal portion of the Aequorea victoria derived venus Yellow Fluorescent Protein gene (nVENUS) and the C-terminal portion of the Aequorea victoria derived venus Yellow Fluorescent Protein gene (cVENUS) for analysis by bimolecular fluorescence complementation. Linker sequences can optionally be included to fuse the target sequence to the paired sequence that enables co-localization. In addition, initiation sequences for strong initiation of translation, such as a Kozak sequence [SEQ ID NO:21] can optionally be included.

As used herein, an “expression plasmid” refers to a nucleic acid construct including elements for the production of a target product in plants. As an example, “expression plasmid” refers to a plasmid including an expression cassette including a target sequence that is operatively linked to an expression regulatory region, a selection sequence that allows for the expression plasmid to be selected for in E. coli, a first origin of replication that allows for the expression plasmid to be propagated in E. coli, a second origin of replication that allows for the expression plasmid to be propagated in either E. coli or Agrobacterium, an initiation sequence for initiation of expression plasmid DNA replication, and a left boundary sequence and a right boundary sequence that are recognized by Agrobacterium and allow for the expression cassette to be transformed into plant cells and integrated into the plant genome.

As used herein, a “vector backbone” refers to a nucleic acid construct including elements necessary for the assembly, propagation, and selection of expression plasmids in bacteria. As an example, “vector backbone” refers to a plasmid including nucleic acid sequences encoding restriction sites that enable assembly of expression plasmids using cloning techniques, an initiation sequence for initiation of expression plasmid DNA replication, a selection sequence that allows for the plasmid to be selected for in E. coli, a first origin of replication that allows for the plasmid to be propagated in E. coli, a second origin of replication that allows for the plasmid to be propagated in either E. coli or Agrobacterium. The vector backbone can also include identification sequences, such as sequences encoding the lacZ gene [SEQ ID NO:22] that is replaced during cloning to aid in identification of desired clones through blue/white screening, and a left boundary sequence and a right boundary sequence that are recognized by Agrobacterium and allow for the expression cassette to be transformed into plant cells and integrated into the plant genome.

In the expression plasmid and vector backbone, examples of selection sequences that allow the plasmid to be selected for in E. coli include sequences for genes encoding antibiotic resistance, and as a specific example includes sequences encoding neomycin phosphotransferase II [SEQ ID NO:23] encoding an aminoglycoside phosphotransferase conferring resistance to kanamycin. Examples of a first origin of replication include any sequence that allows for the plasmid to be propagated in E. coli, and as a specific example includes sequences for a pUC origin of replication [SEQ ID NO:24]. Examples of a second origin of replication include any sequence that allows for the plasmid to be propagated in either E. coli or Agrobacterium, and as a specific example includes sequences for an oriV origin of replication [SEQ ID NO:25] for high copy expression in E. coli or Agrobacterium. Examples of initiation sequences include any sequence that enables initiation of plasmid DNA replication, and as a specific example includes sequences encoding the TrfA replication initiation protein [SEQ ID NO:26]. Examples of left boundary sequences and right boundary sequences include any sequences that are recognized by Agrobacterium and allow for transformation into plant cells and integration into the plant genome, and as specific examples include repeated sequences from nopaline C58 T-DNA [SEQ ID NO:27]. Examples of restriction sites include any sequences that enable assembly of expression plasmids using cloning techniques, and as specific examples include BsaI restriction sites that allow for cloning of a single expression cassette into a vector backbone using GoldenGate or MoClo methods.

As used herein, the term “plant” includes reference to whole plant, plant organ, plant tissues, and plant cell and progeny of same, but is not limited to angiospems and gymnosperms such as Arabidopsis, potato, tomato, tobacco, alfalfa, lemice, carrot, strawberry, sugarbeet, cassava, sweet potato, soybean, lima bean, pea, chick pea, maize (corn), turf grass, wheat, rice, barley, sorghum, oat, oak, eucalyptus, walnut, palm and duckweed a well as fern and moss. Thus, a plant may be a monocot, a dicot, a vascular plant reproduced from spores such as fern or a nonvascular plant such as moss, liverwort, hornwort and algae. The term “plant,” as used herein, also encompasses plant cells, seeds, plant progeny, propagule whether generated sexually or asexually, and descendants of any of these, such as cuttings or seed. Plant cells include suspension cultures, callus, embryos, meristematic regions, callus tissue, leaves, roots, shoots, gametophytes, sporophytes, pollen, seeds and microspores. Plants may be at various stages of maturity and may be grown in liquid or solid culture, or in soil or suitable media in pots, greenhouses or fields.

As used herein, the term “dicot” refers to a flowering plant whose embryos have two seed leaves or cotyledons. Examples of dicots include Arabidopsis, tobacco, tomato, potato, sweet potato, cassava, alfalfa, lima bean, pea, chick pea, soybean, carrot, strawberry, lettuce, oak, maple, walnut, rose, mint, squash, daisy, quinoa, buckwheat, mung bean, cow pea, lentil, lupin, peanut, fava bean, French beans, mustard, or cactus.

As used herein, the term “monocot” refers to a flowering plant whose embryos have one cotyledon or seed leaf. Examples of monocots include turf grass, maize (corn), rice, oat, wheat, barley, sorghum, orchid, iris, lily, onion, palm, and duckweed.

As used herein, the term “transgenic plant” means a plant that has been transformed with one or more exogenous nucleic acids. “Transformation” refers to a process by which a nucleic acid is stably integrated into the genome of a plant cell. “Stably transformed” refers to the permanent, or non-transient, retention, expression, or a combination thereof of a polynucleotide in and by a cell genome. A stably integrated polynucleotide is one that is a fixture within a transformed cell genome and can be replicated and propagated through successive progeny of the cell or resultant transformed plant. Transformation can occur under natural or artificial conditions using various methods. Transformation can rely on any method for the insertion of nucleic acid sequences into a prokaryotic or eukaryotic host cell, including Agrobacterium-mediated transformation as illustrated in U.S. Pat. Nos. 5,159,135; 5,824,877; 5,591,616 and 6,384,301, all of which are incorporated herein by reference in its entirety. Methods for plant transformation also include microprojectile bombardment as illustrated in U.S. Pat. Nos. 5,015,580; 5,550,318; 5,538,880; 6,153,812; 6,160,208; 6,288,312 and 6,399,861, all of which are incorporated herein by reference in its entirety. Recipient cells for the plant transformation include meristem cells, callus, immature embryos, hypocotyls explants, cotyledon explants, leaf explants, and gametic cells such as microspores, pollen, sperm and egg cells, and any cell from which a fertile plant can be regenerated, as described in U.S. Pat. Nos. 6,194,636; 6,232,526; 6,541,682 and 6,603,061 and U.S. Patent Application publication US 2004/0216189 A1, all of which are incorporated herein by reference in its entirety.

As used herein, the term “stably expressed” refers to expression and accumulation of a protein in a plant cell over time. As an example, a recombinant protein may accumulate because it is not degraded by endogenous plant proteases. As a further example, a recombinant protein is considered to be stably expressed in a plant if it is present in the plant in an amount of 1% or higher per total protein weight of soluble protein extractable from the plant.

As used herein, the term “recombinant” refers to nucleic acids or proteins formed by laboratory methods of genetic recombination (e.g., molecular cloning) to bring together genetic material from multiple sources, creating sequences that would otherwise not be found in the genome. Recombinant proteins may be expressed in vivo in various types of host cells, including plant cells, bacterial cells, fungal cells, avian cells, and mammalian cells. Recombinant proteins may also be generated in vitro.

A specified nucleic acid is “derived from” a given nucleic acid when it is constructed using the given nucleic acid's sequence, or when the specified nucleic acid is constructed using the given nucleic acid. For example, a cDNA or EST is derived from an expressed mRNA.

The recombinant proteins described herein may comprise one or more egg white proteins. As used herein, the term “egg white protein” refers to any protein, or fragment or variant thereof, that is typically found in avian eggs. In some cases, the egg white protein is at least one of ovalbumin, ovotransferrin, ovomucoid, ovoglobulin g2, ovoglobulin g3, ovomucin, lysozyme, ovoinhibitor, ovoglycoprotein, flavoprotein, ovomacroglobulin, avidin, or cystati. In some cases, the egg white protein does not comprise avidin. In some cases, the origin of the egg white protein is a bird species, for example, chicken, ducks, turkey, goose, guinea hen, gull, quails, pheasant, ostrich, or emu. In some instances, the egg is produced by a domestic bird, including, for example, chickens, turkeys, pigeons, quails, geese, and ducks. Examples of recombinant proteins include any Gallus gallus egg white protein expressed in a plant, and as specific examples include recombinant Gallus gallus ovalbumin [SEQ ID NO: 28] and recombinant Gallus gallus ovomucoid [SEQ ID NO:29].

As used herein, the term “tagged protein” refers to a recombinant protein that includes additional peptides that are not part of the native protein and that remain after post-translational processing. Examples of tagged proteins include any recombinant protein including additional peptides after post-translational processing in a plant cell, and as specific examples include recombinant Gallus gallus ovalbumin produced in a plant transformed with pMOZ183 [SEQ ID NO:32] or pMOZ243 [SEQ ID NO:35]. Specific examples also include recombinant Gallus gallus ovomucoid produced in a plant transformed with pMOZ184 [SEQ ID NO:33], pMOZ246 [SEQ ID NO:34], or pMOZ244 [SEQ ID NO:36].

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, the singular forms “a”, “an” and “the” are intended to include the plural forms as well, unless the context clearly indicates otherwise. Furthermore, to the extent that the terms “including”, “includes”, “comprising”, “has”, “with”, or variants thereof are used in either the detailed description and/or the claims, such terms are intended to be inclusive in a manner similar to the term “comprising.”

As used herein, the term “by-product” in a food composition refers to an ingredient introduced to the food composition during manufacture of the food composition.

As used herein, the term “a detectable amount” refers to an amount of a composition (e.g., a molecule) that can be detected using the most sensitive analytical techniques up to date, including for example, liquid chromatography methods (e.g., reverse phase HPLC, size exclusion, normal phase chromatography), mass spectrometry (e.g., electrospray tandem mass spectrometry, and electrospray FT-ICR mass spectrometry), or a combination of analytical techniques (e.g., liquid chromatography-tandem mass spectrometry (LC-MS/MS)). In some cases, a detectable amount is at a concentration above 10⁻² mol/L, 10⁻³ mol/L, 10⁻⁴ mol/L, 10⁻⁵ mol/L, 10⁻⁶ mol/L, 10⁻⁷ mol/L, 10⁻⁸ mol/L, 10⁻⁹ mol/L, or 10⁻¹⁰ mol/L.

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

1. Construction of pMOZ Expression Plasmids for Plant Transformation

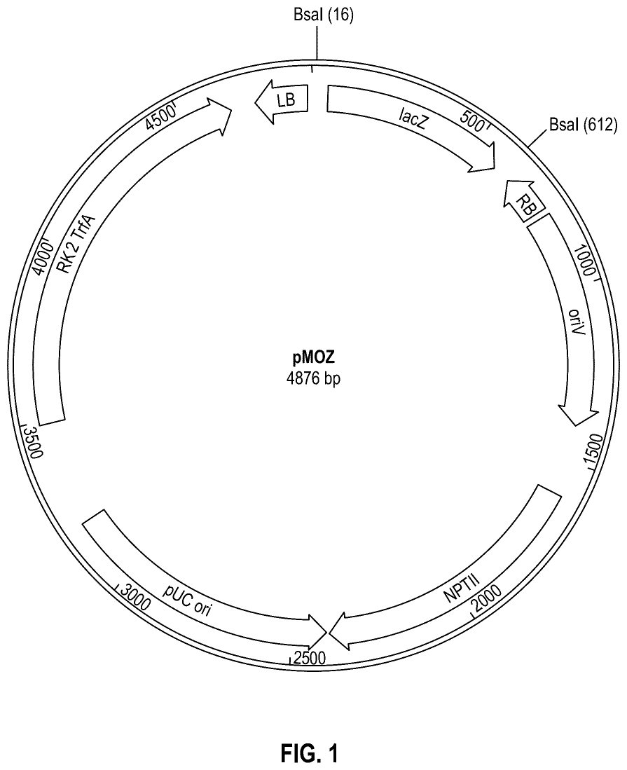

Referring now to FIG. 1, therein is shown an example of a pMOZ vector backbone [SEQ ID NO:30], which is an example vector backbone used to construct pMOZ expression plasmids for the production of recombinant egg white proteins in plants. As a specific example, FIG. 1 is a map of a pMOZ vector backbone including a selection sequence encoding a neomycin phosphotransferase II gene (NPTII) that encodes an aminoglycoside phosphotransferase conferring resistance to kanamycin that allows for the plasmid to be selected for in E. coli, a first origin of replication (pUC ori) [SEQ ID NO:24] that allows for the plasmid to be propagated in E. coli, and a second origin of replication (oriV) [SEQ ID NO:25] that allows for the plasmid to be propagated in either E. coli or Agrobacterium. In this way, pMOZ expression plasmids can be assembled using cloning methods in bacteria and then transferred to Agrobacterium for transformation into plants.

Continuing this example, the pMOZ vector backbone further includes two BsaI restriction sites (BsaI) that allow for cloning of a single expression cassette into the pMOZ vector backbone using GoldenGate or MoClo methods, an identification sequence encoding the lacZ gene (lacZ) [SEQ ID NO:22] to aid in the identification of correct clones through E. coli colony blue/white screening, and an initiation sequence encoding the TrfA replication initiation protein (RK2 TrfA) [SEQ ID NO: 26] for initiation of plasmid DNA replication. The BsaI sites are flanked by a left border repeat (LB) and a right border repeat (RB) from nopaline C58 T-DNA [SEQ ID NO:16] that are recognized by Agrobacterium and allow for an expression cassette to be transformed into plant cells and integrated into the plant host genome.

Further continuing this example, the pMOZ vector backbone was constructed by cloning a synthesized 640 bp double stranded DNA fragment [SEQ ID NO:31] into plasmid pAGM467 (available through Addgene, plasmid 48014). The synthesized 640 bp double stranded DNA fragment includes sequences encoding the lacZ gene [SEQ ID NO:22] to aid in identification of desired clones through blue/white screening.

Further continuing this example, the cloning of the pMOZ vector backbone was done by mixing pAGM4673 and the synthetic fragment in a 1:3 molar ratio in the presence of BbsI (New England Biolabs, product R3539S) and T4 ligase (New England Biolabs, product M0202S). The mixture was incubated in a thermocycler using the following program (37° C., 60 s; 16° C. 120 s; repeat 30×), and used to transform competent E. coli (Lucigen, 60117-1). E. coli were grown on Luria-Bertani agar plates with 50 μg/mL kanamycin, and the pMOZ vector backbone was purified from a single colony. The purified pMOZ vector backbone was Sanger sequenced to confirm the plasmid was assembled as planned.

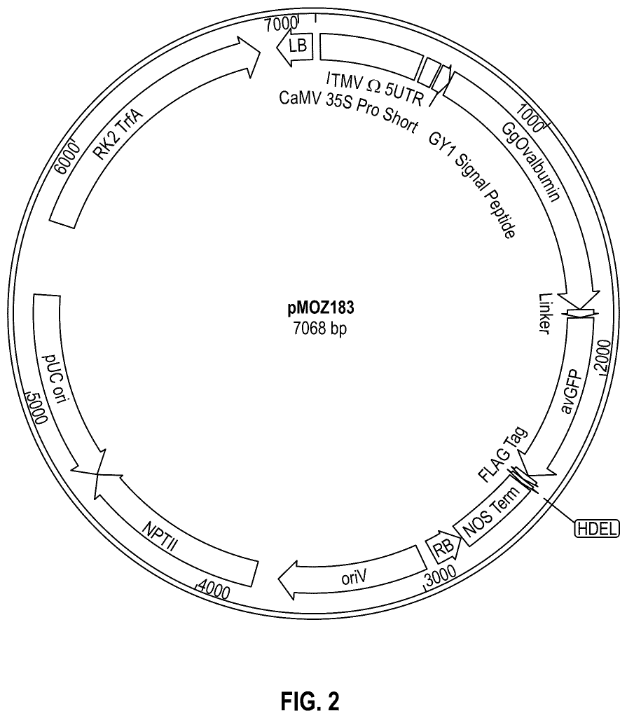

Referring now to FIG. 2, therein is shown an example of a pMOZ expression plasmid for the production of recombinant Gallus gallus egg white proteins in plants constructed using the pMOZ vector backbone shown in FIG. 1 using GoldenGate or MoClo methods. As a specific example, FIG. 2 is a map of a pMOZ183 expression plasmid [SEQ ID NO:32], which is an example expression plasmid for the production of recombinant Gallus gallus ovalbumin:GFP:FLAG in plants. Continuing this example, the pMOZ183 expression plasmid includes a 1158 bp target sequence encoding Gallus gallus ovalbumin (GgOvalbumin) [SEQ ID NO:5] that had been synthesized using only coding sequences from the original Gallus gallus ovalbumin gene and was codon optimized for expression in plants using Nicotiana benthamiana as the model expression system, operatively linked to a 426 bp sequence encoding the Cauliflower Mosaic Virus 35S promoter (CaMV 35S Pro Short) [SEQ ID NO:1], a 62 bp translational enhancer encoding the omega leader sequence of the Tobacco Mosaic Virus (TMV Ω 5UTR) [SEQ ID NO:2], and a 57 bp N-terminal localization sequence encoding the soybean GY1 signal peptide (GY1 Signal Peptide) [SEQ ID NO:7] for localizing the recombinant protein to the endoplasmic reticulum of the plant cell where the recombinant protein can accumulate and not be degraded by the proteasome.

Further continuing this example, the pMOZ183 expression plasmid also includes a 717 bp label sequence encoding the Aequorea victoria Green Fluorescent Protein gene (avGFP) that had been synthesized using only the coding sequences of the original A. victoria Green Fluorescent Protein gene to create a visual marker to identify the recombinant protein by fluorescence microscopy, a 33 bp linker sequence (Linker) to fuse the target sequence to the label sequence, a 24 bp sequence encoding the peptide DDDDK (FLAG Tag) [SEQ ID NO: 48] for detection of the recombinant protein by Western blot analysis, a 12 bp retention sequence encoding the peptide HDEL (SEQ ID NO: 37) (which in some instances can be coded by DNA sequence SEQ ID NO:10]) that retains the recombinant protein in the endoplasmic reticulum of plant cells, a 263 bp terminator sequence encoding nopaline synthase terminator (NOS Term) [SEQ ID NO:3] that serves as a universal termination sequence for plant gene expression and can increase mRNA stability through polyadenylation, a right border repeat (RB) and left border repeat (LB) from nopaline C58 T-DNA [SEQ ID NO:27] for transgene integration into the plant host genome, a first origin of replication (pUC ori) [SEQ ID NO:24], a second origin of replication (oriV) [SEQ ID NO:14], an initiation sequence encoding the TrfA replication initiation protein (RK2 TrfA) [SEQ ID NO:26], and a selection sequence encoding neomycin phosphotransferase II (NPTII) [SEQ ID NO:23] encoding an aminoglycoside phosphotransferase conferring resistance to kanamycin.

2. Expression of Gallus gallus Ovalbumin in Plants

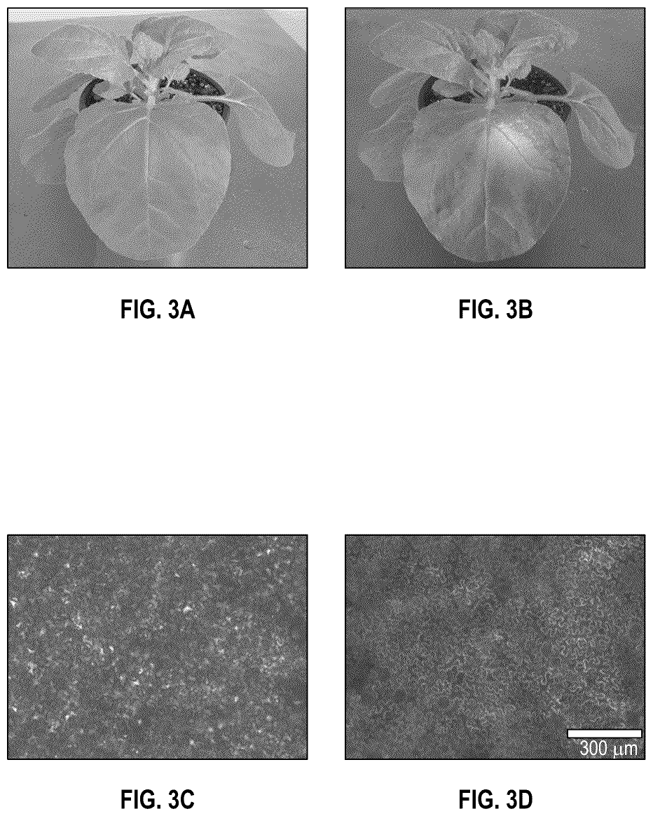

Referring now to FIGS. 3A-D, therein is shown an example of the expression of recombinant ovalbumin:GFP:FLAG in a systemically-infected Nicotiana benthamiana plant using the pMOZ183 expression plasmid [SEQ ID NO:32] in FIG. 2. In this example, Nicotiana benthamiana plants were incubated in a growth room at 25° C. with a 16 hour light 8 hour dark cycle for 4 weeks. The 4-week old Nicotiana benthamiana plants were infiltrated with Agrobacterium tumefaciens strain GV3101 carrying pMOZ183 at an OD₆₀₀ of 0.1. Following vacuum infiltration, plants were blot-dried and returned to the growth room for 72 hours before being imaged. Images show expression of recombinant ovalbumin:GFP:FLAG from pMOZ183 in a systemically infected Nicotiana benthamiana plant photographed under ambient white light (FIG. 3A), and under UV light (FIG. 3B), and in leaf epidermal cells photographed using an epifluorescence microscope at 10× magnification with brightfield (FIG. 3C) and with Green Fluorescent Protein (GFP) excitation and emission filters (FIG. 3D).

Continuing this example, Nicotiana benthamiana plants transformed with the pMOZ183 expression plasmid remained green and free from tissue necrosis (FIG. 3A); the majority of leaf tissue was fluorescent when viewed without a microscope using a GFP-emitting light at 440-460 nm with the corresponding 500-560 nm bandpass filter (FIG. 3B); leaf epidermal cells were intact and turgid when viewed with an epifluorescence microscope by brightfield (FIG. 3C); and the majority of leaf epidermal cells were fluorescent when viewed with epifluorescence microscope with GFP excitation and emission filters (FIG. 3D), with similar results obtained in over 500 leaf tissue sections from 3 different plants. These data show that the recombinant Gallus gallus egg white protein ovalbumin can be produced in Nicotiana benthamiana plants using the pMOZ expression system without the need for co-expression of other Gallus gallus egg white proteins.

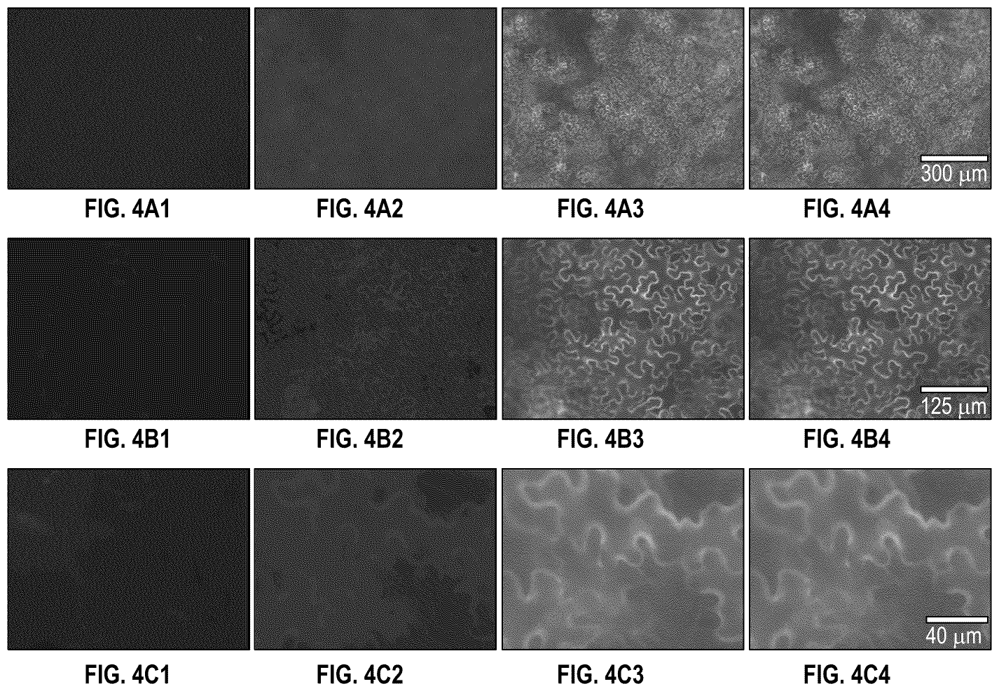

Referring now to FIGS. 4A1-4C4, therein is shown a further example of the expression of recombinant ovalbumin:GFP:FLAG in a systemically-infected Nicotiana benthamiana plant using the pMOZ183 expression plasmid [SEQ ID NO:32] in FIG. 2. The majority of leaf epidermal cells were fluorescent when viewed with epifluorescence microscope with Green Fluorescent Protein (GFP) excitation and emission filters at 10× magnification (FIG. 4A3), at 20× magnification (FIG. 4B3), and at 60× magnification (FIG. 4C3), but were not fluorescent when viewed at 20×, 40×, or 60× magnification with Red Fluorescent Protein (RFP) excitation and emission filters (FIGS. 4A1, 4B1, 4C1) or Yellow Fluorescent Protein (YFP) excitation and emission filters (FIGS. 4A2, 4B2, 4C2); and no additional fluorescence was observed when the images from all filters were superimposed (FIGS. 4A4, 4B4, 4C4), with similar results obtained in over 500 leaf tissue sections from 3 different plants. These data further show that the recombinant Gallus gallus egg white protein ovalbumin can be produced in Nicotiana benthamiana plants using the pMOZ expression system without the need for co-expression of other Gallus gallus egg white proteins.

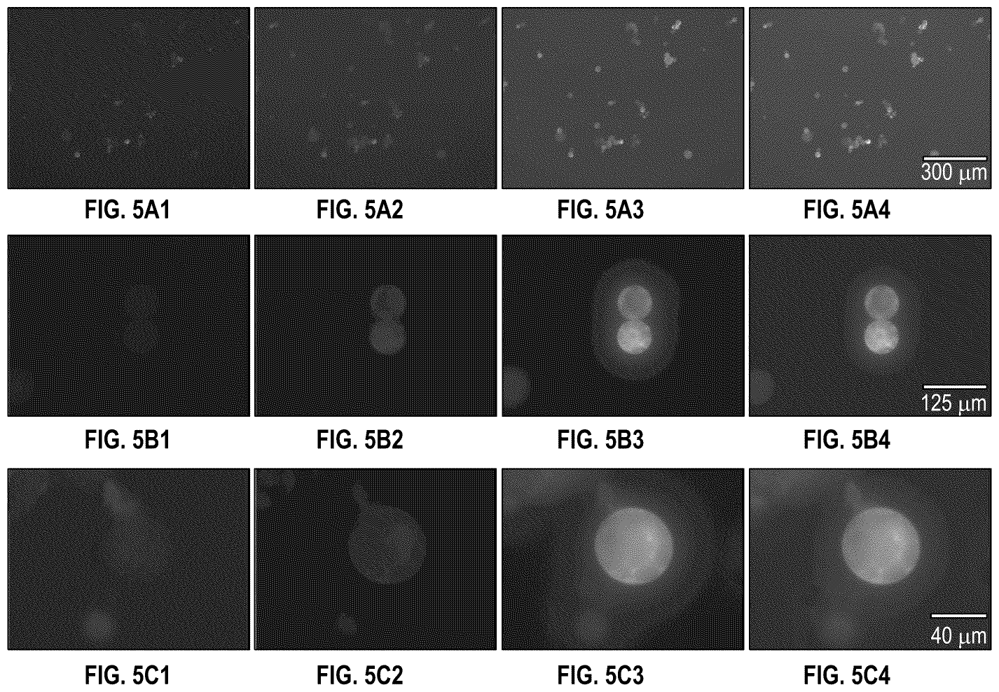

Referring now to FIGS. 5A1-5C4, therein is shown an example of the expression of recombinant ovalbumin:GFP:FLAG in Glycine max protoplasts transformed with the pMOZ183 expression plasmid [SEQ ID NO:32] in FIG. 2. In this example, protoplasts derived from embryonic cotyledons of immature Glycine max plants were transformed with the pMOZ183 expression plasmid using polyethylene glycol, and photographed using an epifluorescence microscope.

Continuing this example, the majority of Glycine max protoplasts were fluorescent when viewed with epifluorescence microscope at 10× magnification with GFP excitation and emission filters (FIG. 5A3), but were not fluorescent when viewed with Red Fluorescent Protein (RFP) excitation and emission filters (FIG. 5A1) or Yellow Fluorescent Protein (YFP) excitation and emission filters (FIG. 5A2); and showed no significant additional fluorescence when the images from all filters were superimposed (FIG. 5A4).

Further continuing this example, the fluorescence observed with GFP excitation and emission filters at 20× magnification (FIG. 5B3) and 60× magnification (FIG. 5C3) was localized to Glycine max protoplasts, while no fluorescence was observed with RFP excitation and emission filters at 20× magnification (FIGS. 5B1, 5C1) or YFP excitation and emission filters (FIGS. 5B2, 5C2); and no significant additional fluorescence was observed when the images from all filters were superimposed (FIGS. 5B4, 5C4). These data show that the Gallus gallus egg white protein ovalbumin can be produced in generally in plant cells using the pMOZ expression system without the need for co-expression of other Gallus gallus egg white proteins.

3. Expression of Gallus gallus Ovomucoid in Plants

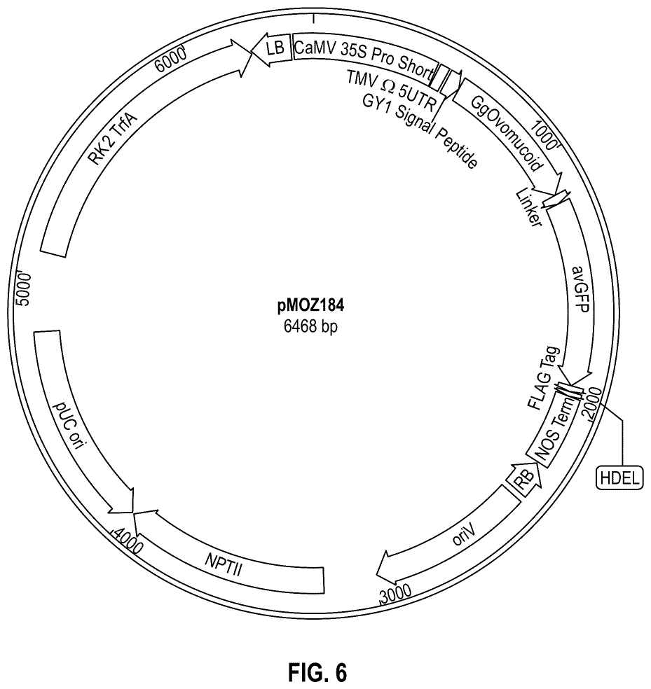

Referring now to FIG. 6, therein is shown an example of a pMOZ expression plasmid for the production of Gallus gallus egg white proteins in plants constructed from the pMOZ vector backbone [SEQ ID NO:30] shown in FIG. 1 using Golden Gate or MoClo methods. As a specific example, FIG. 6 is a map of a pMOZ184 expression plasmid [SEQ ID NO:33], which is an example expression plasmid for the production of recombinant ovomucoid:GFP:FLAG in plants according to the present disclosure. Continuing this example, the pMOZ184 expression plasmid includes a 558 bp target sequence encoding Gallus gallus ovomucoid (GgOvomucoid) [SEQ ID NO:29] that had been synthesized using only coding sequences from the original Gallus gallus ovomucoid gene and was codon optimized for expression in plants using Nicotiana benthamiana as the model expression system, operatively linked to a 426 bp sequence encoding the promoter Cauliflower Mosaic Virus 35S promoter (CaMV 35S Pro Short) [SEQ ID NO:1], a 62 bp translational enhancer encoding the omega leader sequence of the Tobacco Mosaic Virus (TMV Ω 5UTR) [SEQ ID NO:2], a 57 bp N-terminal localization sequence encoding the soybean GY1 signal peptide (GY1 Signal Peptide) [SEQ ID NO:7] for localizing the recombinant protein to the endoplasmic reticulum of the plant cell where the recombinant protein can accumulate and not be degraded by the proteasome.

Further continuing this example, the pMOZ184 expression plasmid also includes a 717 bp label sequence encoding the Aequorea victoria Green Fluorescent Protein gene (avGFP) that had been synthesized using only the coding sequences of the original A. victoria Green Fluorescent Protein gene to create a visual marker to identify the recombinant protein by fluorescence microscopy, a 33 bp linker sequence (Linker) to fuse the target sequence to the label sequence, a 24 bp detection nucleic acid sequence encoding the peptide DDDDK (FLAG Tag) [SEQ ID NO: 48] for detection of the recombinant protein by Western blot analysis, a 12 bp retention sequence encoding the peptide HDEL (SEQ ID NO: 37) (which in some instances can be coded by DNA sequence SEQ ID NO:10]) that retains the recombinant protein in the endoplasmic reticulum of plant cells, a 263 bp terminator sequence encoding nopaline synthase terminator (NOS Term) [SEQ ID NO:3] that serves as a universal termination sequence for plant gene expression and can increase mRNA stability through polyadenylation, a right border repeat (RB) and left border repeat (LB) from nopaline C58 T-DNA [SEQ ID NO:27] for transgene integration into the plant host genome, a first origin of replication (pUC ori) [SEQ ID NO:24], a second origin of replication (oriV) [SEQ ID NO:25], an initiation sequence encoding the TrfA replication initiation protein (RK2 TrfA) [SEQ ID NO:26], and a selection sequence encoding neomycin phosphotransferase II (NPTII) [SEQ ID NO:23] encoding an aminoglycoside phosphotransferase conferring resistance to kanamycin.

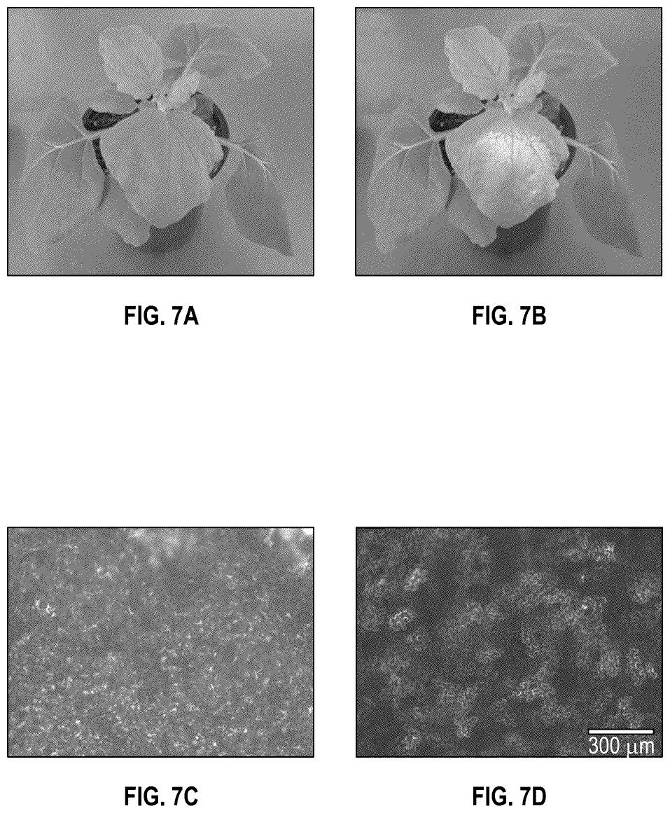

Referring now to FIGS. 7A-D, therein is shown an example of the expression of recombinant ovomucoid:GFP:FLAG in a systemically-infected Nicotiana benthamiana plant using the pMOZ184 expression plasmid [SEQ ID NO:33] in FIG. 6. In this example, Nicotiana benthamiana plants were incubated in a growth room at 25° C. with a 16 hour light 8 hour dark cycle for 4 weeks. The 4-week old Nicotiana benthamiana plants were infiltrated with Agrobacterium tumefaciens strain GV3101 carrying pMOZ184 at an OD₆₀₀ of 0.1. Following vacuum infiltration, plants were blot-dried and returned to the growth room for 72 hours before being imaged. Images show expression of ovomucoid:GFP:FLAG from pMOZ184 in a systemically infected Nicotiana benthamiana plant photographed under ambient white light (FIG. 7A), and under UV light (FIG. 7B), and in leaf epidermal cells photographed using an epifluorescence microscope at 10× magnification with brightfield (FIG. 7C) and with Green Fluorescent Protein (GFP) excitation and emission filters (FIG. 7D).

Continuing this example, Nicotiana benthamiana plants transformed with the pMOZ184 expression plasmid remained green and free from tissue necrosis (FIG. 7A); the majority of leaf tissue was fluorescent when viewed without a microscope using a GFP-emitting light at 440-460 nm with the corresponding 500-560 nm bandpass filter (FIG. 7B); leaf epidermal cells were intact and turgid when viewed with an epifluorescence microscope by brightfield (FIG. 7C); and the majority of leaf epidermal cells were fluorescent when viewed with epifluorescence microscope with GFP excitation and emission filters (FIG. 7D), with similar results obtained in over 500 leaf tissue sections from 3 different plants. These data show that the recombinant Gallus gallus egg white protein ovomucoid can be produced in Nicotiana benthamiana plants using the pMOZ expression system without the need for co-expression of other Gallus gallus egg white proteins.

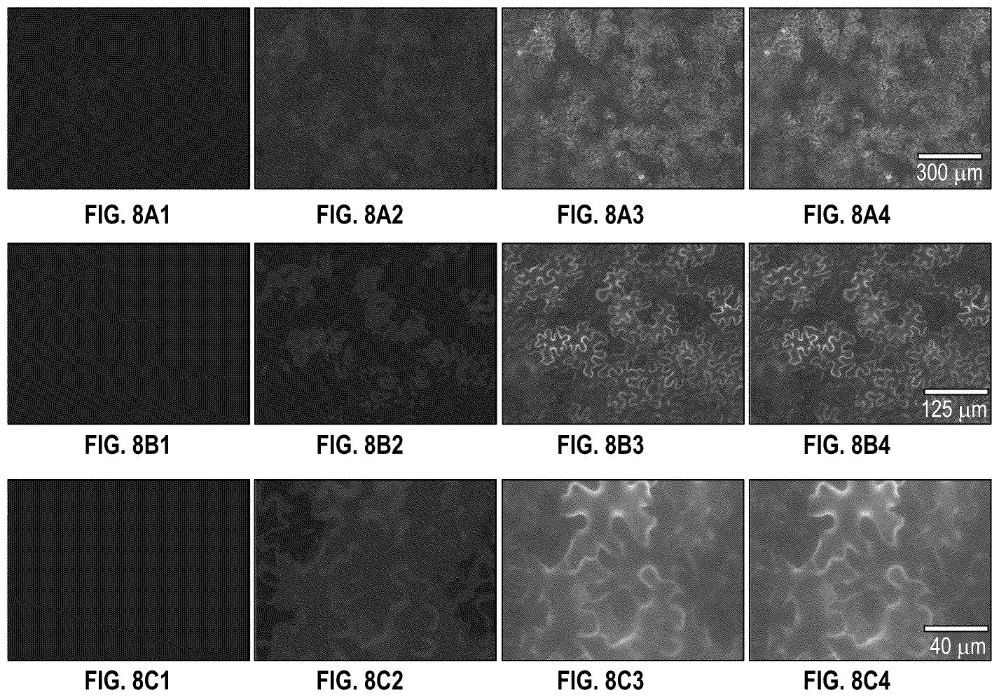

Referring now to FIGS. 8A1-8C4, therein is shown a further example of the expression of recombinant ovomucoid:GFP:FLAG in a systemically-infected Nicotiana benthamiana plant using the pMOZ184 expression plasmid [SEQ ID NO:33] in FIG. 6. In this example, the majority of leaf epidermal cells were fluorescent when viewed with epifluorescence microscope with Green Fluorescent Protein (GFP) excitation and emission filters at 10× magnification (FIG. 8A3), at 20× magnification (FIG. 8B3), and at 60× magnification (FIG. 8C3), but were not fluorescent when viewed at 20×, 40×, or 60× magnification with Red Fluorescent Protein (RFP) excitation and emission filters (FIGS. 8A1, 8B 1, 8C1) or Yellow Fluorescent Protein (YFP) excitation and emission filters (FIGS. 8A2, 8B2, 8C2); and no additional fluorescence was observed when the images from all filters were superimposed (FIGS. 8A4, 8B4, 8C4), with similar results obtained in over 500 leaf tissue sections from 3 different plants. These data further show that the recombinant Gallus gallus egg white protein ovomucoid can be produced in Nicotiana benthamiana plants using the pMOZ expression system without the need for co-expression of other Gallus gallus egg white proteins.

4. Co-Expression of Gallus gallus Ovalbumin and Ovomucoid in Plants

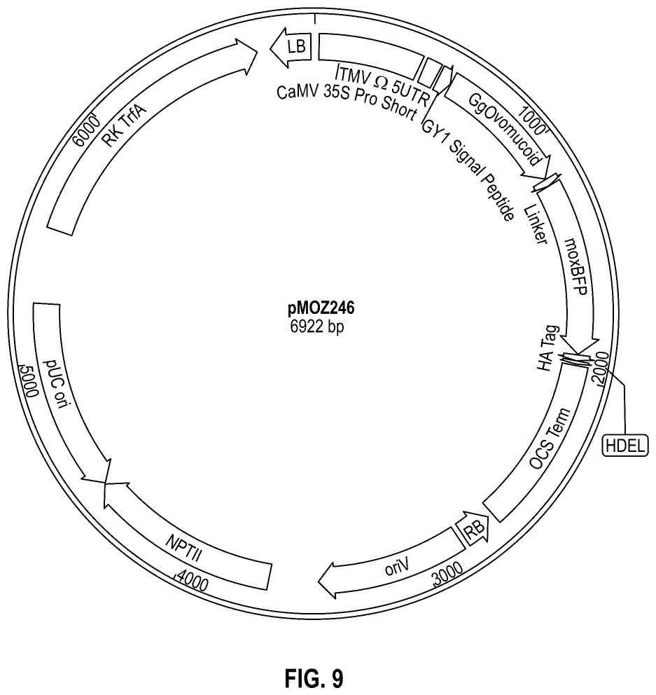

Referring now to FIG. 9, therein is shown an example of a pMOZ expression plasmid for the production of recombinant Gallus gallus egg white proteins in plants constructed from the pMOZ vector backbone [SEQ ID NO:30] shown in FIG. 1 using GoldenGate or MoClo methods. As a specific example, FIG. 9 is a map of a pMOZ246 expression plasmid [SEQ ID NO:34], which is an example expression plasmid for the production of recombinant ovomucoid:BFP:HA in plants. Continuing this example, the pMOZ246 expression plasmid includes a 558 bp target sequence encoding Gallus gallus ovomucoid (GgOvomucoid) [SEQ ID NO:29] that had been synthesized using only coding sequences from the original Gallus gallus ovomucoid gene and was codon optimized for expression in plants using Nicotiana benthamiana as the model expression system, operatively linked to a 426 bp sequence encoding the promoter Cauliflower Mosaic Virus 35S promoter (CaMV 35S Pro Short) [SEQ ID NO:1], a 62 bp translational enhancer encoding the omega leader sequence of the Tobacco Mosaic Virus (TMV Ω 5UTR) [SEQ ID NO:2], and a 57 bp N-terminal localization sequence encoding the soybean GY1 signal peptide gene (GY1 Signal Peptide) [SEQ ID NO:7] for localizing the recombinant protein to the endoplasmic reticulum of the plant cell where the recombinant protein can accumulate and not be degraded by the proteasome.

Further continuing this example, the pMOZ246 expression plasmid also includes a 717 bp label sequence encoding the Aequorea victoria mox Blue Fluorescent Protein (moxBFP) that had been synthesized using only the coding sequences of the original gene to create a visual marker to identify the recombinant protein by fluorescence microscopy, a 30 bp linker sequence (Linker) to fuse the target sequence to the label sequence, a 27 bp sequence encoding the peptide for Hemagglutinin (HA Tag) [SEQ ID NO:20] for detection of the recombinant protein by Western blot analysis, a 12 bp retention sequence encoding the peptide HDEL (SEQ ID NO: 37) (which in some instances can be coded by DNA sequence SEQ ID NO:10]) that retains the recombinant protein in the endoplasmic reticulum of plant cells, a 714 bp terminator sequence encoding octopine synthase terminator (OCS Term) [SEQ ID NO: 4] that serves as a universal termination sequence for plant gene expression and can increase mRNA stability through polyadenylation, a right border repeat (RB) and left border repeat (LB) from nopaline C58 T-DNA [SEQ ID NO: 27] for transgene integration into the plant host genome, a first origin of replication (pUC ori) [SEQ ID NO:24], a second origin of replication (oriV) [SEQ ID NO:25], an initiation sequence encoding the TrfA replication initiation protein (RK2 TrfA) [SEQ ID NO:26], and a selection sequence encoding neomycin phosphotransferase II (NPTII) [SEQ ID NO: 23] encoding an aminoglycoside phosphotransferase conferring resistance to kanamycin.

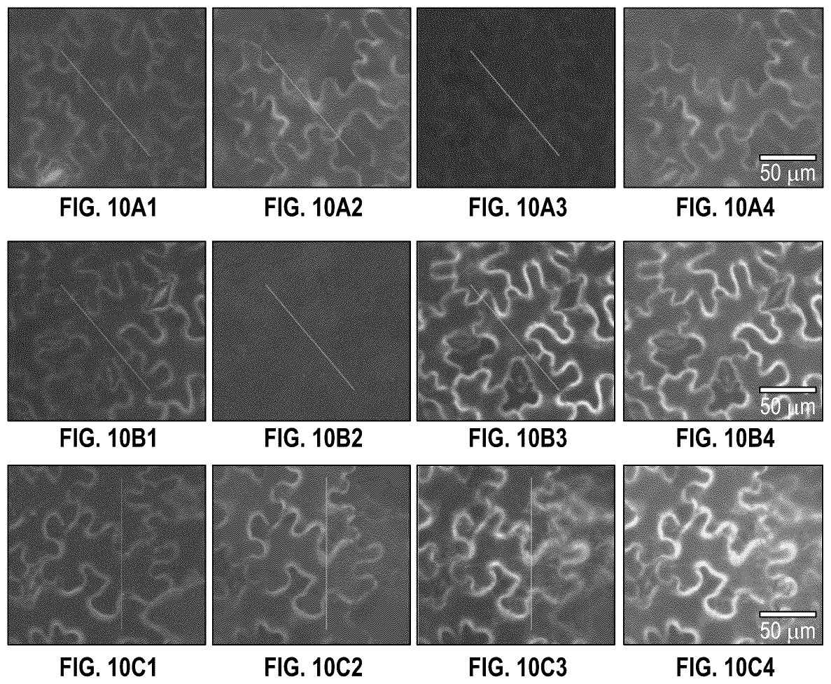

Referring now to FIGS. 10A1-10A4, therein is shown an example of the expression of recombinant ovalbumin:GFP:FLAG in a systemically-infected Nicotiana benthamiana plant using the pMOZ183 expression plasmid [SEQ ID NO:32] in FIG. 2. In this example, Nicotiana benthamiana plants were incubated in a growth room at 25° C. with a 16 hour light 8 hour dark cycle for 4 weeks. The 4-week old Nicotiana benthamiana plants were infiltrated with Agrobacterium tumefaciens strain GV3101 carrying pMOZ183 at an OD₆₀₀ of 0.1. Following vacuum infiltration, plants were blot-dried and returned to the growth room for 72 hours before being imaged. Tissue sections from the systemically-infected Nicotiana benthamiana plants were stained with propidium iodide to visualize the leaf epidermal cell walls.

Continuing this example, the majority of leaf epidural cells were fluorescent when viewed with an epifluorescence microscope with Green Fluorescent Protein (GFP) excitation and emission filters (FIG. 10A2) but were not fluorescent when viewed with Blue Fluorescent Protein (BFP) excitation and emission filters (FIG. 10A3). Further continuing this example, the leaf epidermal cells stained with propidium iodide viewed with Red Fluorescent Protein (RFP) excitation and emission filters showed no signs of cell damage (FIG. 10A1), and matching localization patterns were observed when the images from all filters were superimposed (FIG. 10A4), with similar results obtained in over 500 leaf tissue sections from 3 different plants. These data show that the fluorescent signal detected with the GFP excitation and emission filters was specific to recombinant ovalbumin:GFP:FLAG from the pMOZ183 expression plasmid [SEQ ID NO:32] and was localized in leaf epidermal cells.

Referring now to FIGS. 10B1-10B4, therein is shown an example of the expression of recombinant ovomucoid:BFP:HA in a systemically-infected Nicotiana benthamiana plant using the pMOZ246 expression plasmid [SEQ ID NO:34] in FIG. 9. In this example, Nicotiana benthamiana plants were incubated in a growth room at 25° C. with a 16 hour light 8 hour dark cycle for 4 weeks. The 4-week old Nicotiana benthamiana plants were infiltrated with Agrobacterium tumefaciens strain GV3101 carrying pMOZ246 at an OD₆₀₀ of 0.1. Following vacuum infiltration, plants were blot-dried and returned to the growth room for 72 hours before being imaged. Tissue sections from the systemically-infected Nicotiana benthamiana plants were stained with propidium iodide to visualize the leaf epidermal cell walls.

Continuing this example, the majority of leaf epidural cells were fluorescent when viewed with an epifluorescence microscope with Blue Fluorescent Protein (BFP) excitation and emission filters (FIG. 10B3) but were not fluorescent when viewed with Green Fluorescent Protein (GFP) excitation and emission filters (FIG. 10B2). Further continuing this example, the leaf epidermal cells stained with propidium iodide viewed with Red Fluorescent Protein (RFP) excitation and emission filters showed no signs of cell damage (FIG. 10B1), and matching localization patterns were observed when the images from all filters were superimposed (FIG. 10B4), with similar results obtained in over 500 leaf tissue sections from 3 different plants. These data show that the fluorescent signal detected with the BFP excitation and emission filters was specific to ovomucoid:BFP:HA from the pMOZ246 expression plasmid and was localized in leaf epidermal cells.

Referring now to FIGS. 10C1-10C4, therein is shown an example of the co-expression of recombinant ovalbumin:GFP:FLAG and recombinant ovomucoid:BFP:HA in a systemically-infected Nicotiana benthamiana plant using the pMOZ183 expression plasmid [SEQ ID NO:32] in FIG. 2 and the pMOZ246 expression plasmid [SEQ ID NO:34] in FIG. 9. In this example, Nicotiana benthamiana plants were incubated in a growth room at 25° C. with a 16 hour light 8 hour dark cycle for 4 weeks. The 4-week old Nicotiana benthamiana plants were infiltrated with a mixture of Agrobacterium tumefaciens strain GV3101 carrying pMOZ183 and Agrobacterium tumefaciens strain GV3101 carrying pMOZ246 at an OD₆₀₀ of 0.1. Following vacuum infiltration, plants were blot-dried and returned to the growth room for 72 hours before being imaged. Tissue sections from the systemically-infected Nicotiana benthamiana plants were stained with propidium iodide to visualize the leaf epidermal cell walls.

Continuing this example, the majority of leaf epidural cells were fluorescent when viewed with an epifluorescence microscope with Green Fluorescent Protein (GFP) excitation and emission filters (FIG. 10C2) and also were fluorescent when viewed with Blue Fluorescent Protein (BFP) excitation and emission filters (FIG. 10C3). Further continuing this example, the leaf epidermal cells stained with propidium iodide viewed with Red Fluorescent Protein (RFP) excitation and emission filters showed no signs of cell damage (FIG. 10C1), and matching localization patterns were observed when the images from all filters were superimposed (FIG. 10B4), with similar results obtained in over 500 leaf tissue sections from 3 different plants. These data show that recombinant ovalbumin:GFP:FLAG from the pMOZ183 expression plasmid and recombinant ovomucoid:BFP:HA from the pMOZ246 expression plasmid are co-localized in leaf epidermal cells.

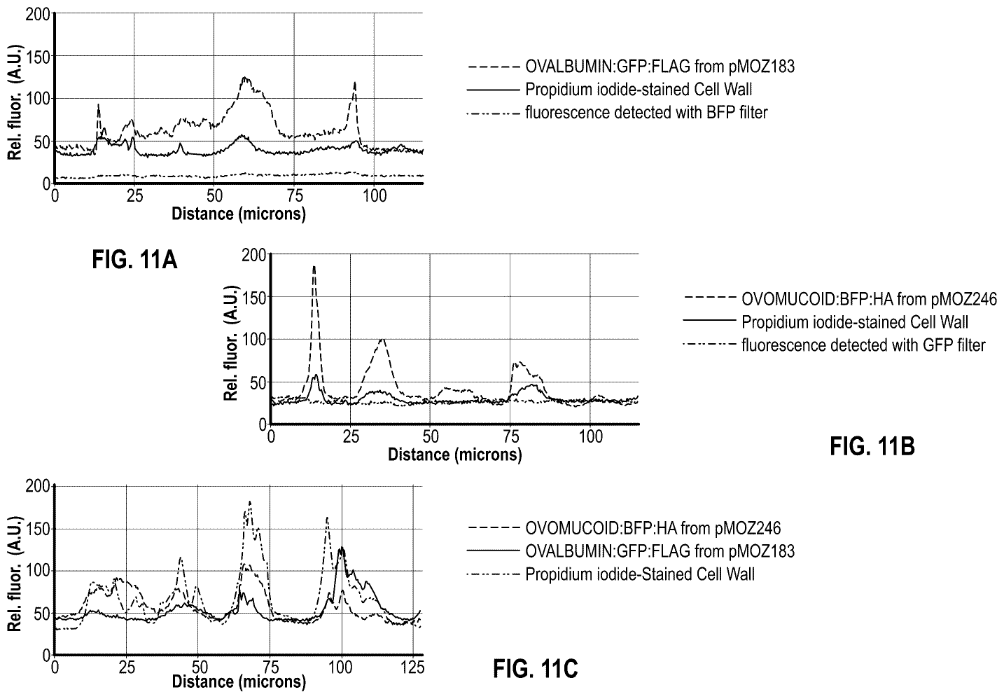

Referring now to FIGS. 11A-11C, therein is shown quantitative analysis of the examples of the expression of recombinant ovalbumin:GFP:FLAG from the pMOZ183 expression plasmid [SEQ ID NO:32] alone (FIG. 11A), recombinant ovomucoid:BFP:HA from the pMOZ246 expression plasmid [SEQ ID NO:34] alone (FIG. 11B), and recombinant ovalbumin:GFP:FLAG and recombinant ovomucoid:BFP:HA from the pMOZ183 and pMOZ246 expression plasmids together (FIG. 11C). In this example, fluorescence intensities from the tissue sections shown in FIG. 10 were measured across a line and plotted to show the relative intensities for propidium iodide-stained leaf epidermal cells, ovalbumin: GFP: FLAG, and ovomucoid:BFP:HA.

Continuing this example, in leaf tissue sections from Nicotiana benthamiana plants transformed with the pMOZ183 expression plasmid alone, peaks were observed across the line measuring fluorescence from propidium iodide-stained leaf epidermal cells (red line) that paralleled the peaks observed from recombinant ovalbumin:GFP:FLAG (green line), while no fluorescence was detected with the BFP excitation and emission filters (blue line) (FIG. 11A).

Further continuing this example, in leaf tissue sections from Nicotiana benthamiana plants transformed with the pMOZ246 expression plasmid alone, peaks were observed across the line measuring fluorescence from propidium iodide-stained leaf epidermal cells (red line) that paralleled the peaks observed from recombinant ovomucoid:BFP:HA (blue line), while no fluorescence was detected with the GFP excitation and emission filters (green line) (FIG. 11B).

Further continuing this example, in leaf tissue sections from Nicotiana benthamiana plants co-transformed with the pMOZ183 and pMOZ246 expression plasmids together, peaks were observed across the line measuring fluorescence from propidium iodide-stained leaf epidermal cells (red line) that paralleled the peaks observed from recombinant ovalbumin:GFP:FLAG (green line) and the peaks from ovomucoid:BFP:HA (blue line) (FIG. 11C). These data further show that recombinant ovalbumin:GFP:FLAG from the pMOZ183 expression plasmid and recombinant ovomucoid:BFP:HA from the pMOZ246 expression plasmid are co-localized in leaf epidermal cells.

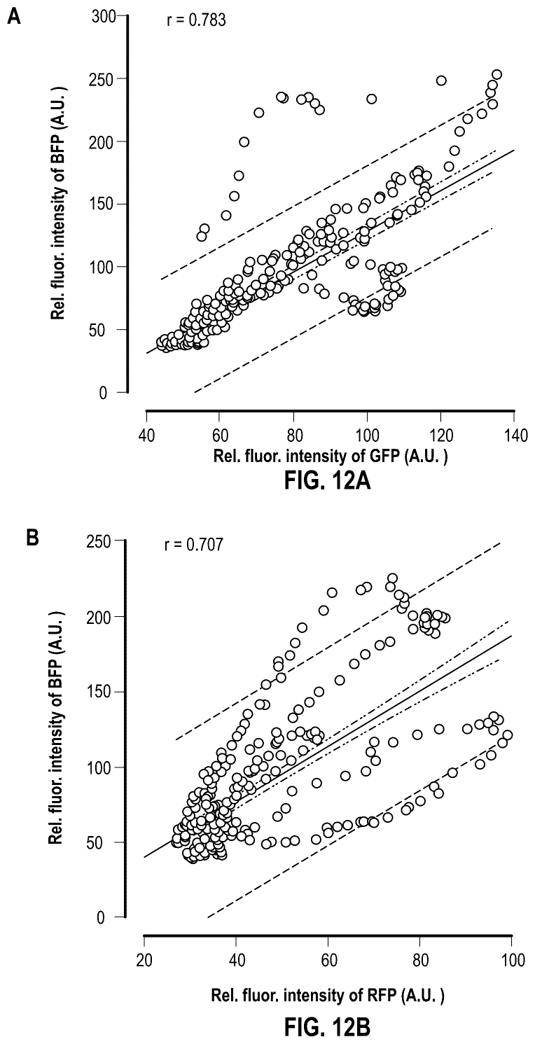

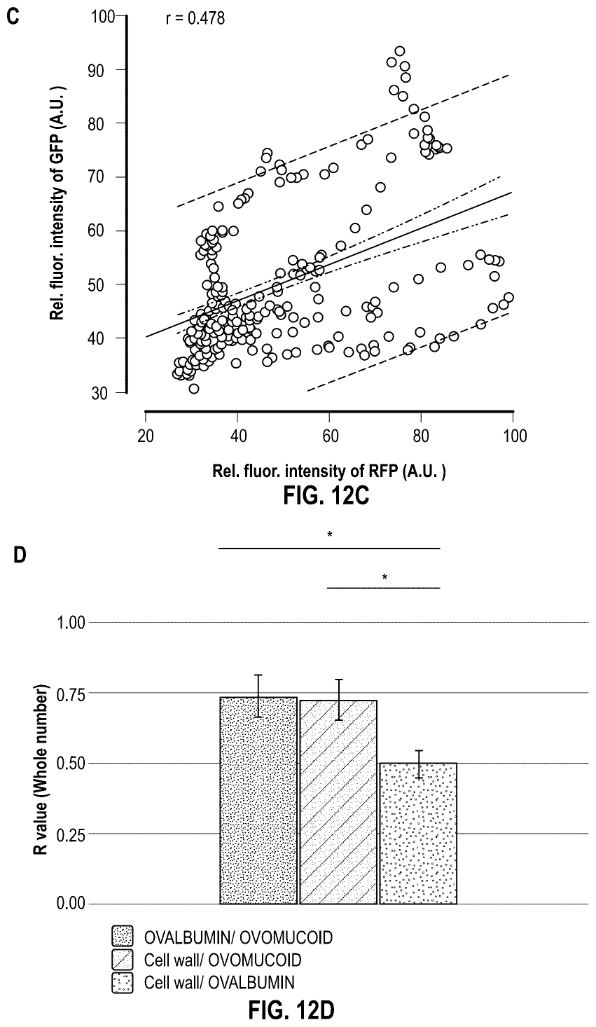

Referring now to FIGS. 12A-12C, therein is shown a Pearson's correlation regression analysis applied to the fluorescence intensities measured for leaf epidermal cell walls, recombinant ovalbumin:GFP:FLAG, and recombinant ovomucoid:BFP:HA in tissue sections from Nicotiana benthamiana plants co-transformed with the pMOZ183 expression plasmid [SEQ ID NO:32] and the pMOZ246 expression plasmid [SEQ ID NO:34] together. In this example, Pearson's correlation regression analysis line plots created using measurements from 9 tissues sections comparing the relative fluorescence intensity measured for ovomucoid:BFP:HA versus ovalbumin:GFP:FLAG (FIG. 12A), ovomucoid:BFP:HA versus leaf epidermal cell walls (FIG. 12B), and ovalbumin:GFP:FLAG versus leaf epidermal cell walls (FIG. 12C) showing r values of 0.783, 0.707, and 0.478, respectively.

Continuing this example, no statistically significant differences were observed between the average r value of ovomucoid:BFP:HA versus ovalbumin:GFP:FLAG and the average r value of ovomucoid:BFP:HA versus ovalbumin:GFP:FLAG (FIG. 12D, showing the average Pearson's correlation r value for all 27 regression analysis, where statistical significance is indicated with a star). These data further show that ovalbumin:GFP:FLAG from the pMOZ183 expression plasmid and ovomucoid:BFP:HA from the pMOZ246 expression plasmid are co-localized in leaf epidermal cells.

5. Colony PCR Analysis of Agrobacterium Carrying pMOZ Expression Plasmids

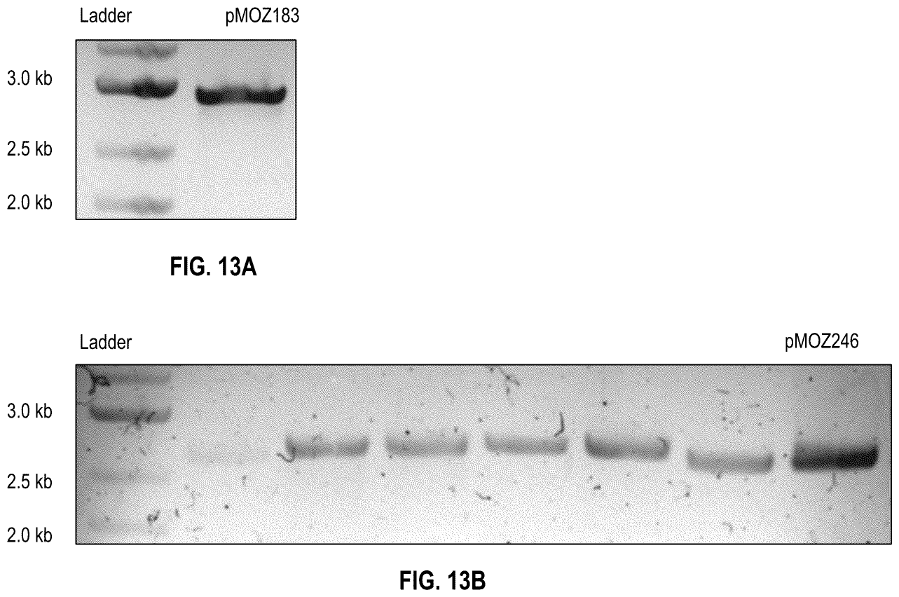

Referring now to FIGS. 13A-13B, therein is shown the results of colony PCR analysis. In this example, colonies of Agrobacterium tumefaciens transformed with the pMOZ183 expression plasmid [SEC ID NO:32] or the pMOZ246 expression plasmid [SEQ ID NO:34] were selected and used as templates for PCR-based genotyping with pMOZ-specific forward primer (5′-atattgtggtgtaaacaaattgacgctt-3′) (SEQ ID NO: 49) and a pMOZ-specific reverse primer (5′-taataaacgctcttttctcttaggtttacc-3′) (SEQ ID NO: 50).

Continuing this example, PCR-based genotyping identified the PCR product of the pMOZ183 expression plasmid at the expected molecular weight of 2.9 kb (FIG. 13A) and the PCR product of the pMOZ246 expression plasmid at the expected molecular weight of 2.75 kb (FIG. 13B). These data show that the expected gene products are produced in Agrobacterium transformed with the pMOZ183 and pMOZ246 expression plasmids.

6. Western Blot and SDS-PAGE of Ovalbumin and Ovomucoid Expressed in Plants

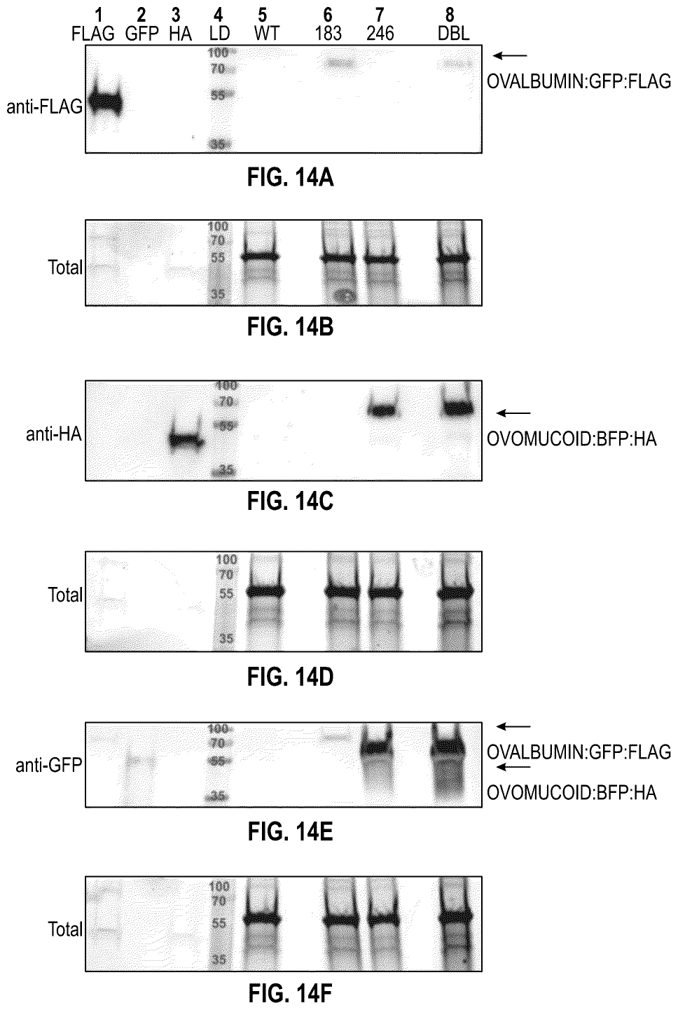

Referring now to FIGS. 14A-14F, therein is shown the results of SDS-PAGE and Western blot analysis showing the detection of recombinant ovalbumin:GFP:FLAG from the pMOZ183 expression plasmid [SEQ ID NO:32] and recombinant ovomucoid:BFP:HA from the pMOZ246 expression plasmid [SEQ ID NO:34] in leaf epidermal cells of systemically infected Nicotiana benthamiana plants that were wild type (WT), plants transformed with the pMOZ183 expression plasmid alone (MZ183), plants transformed with the pMOZ246 expression plasmid alone (MZ246), and plants co-transformed with the pMOZ183 and the pMOZ246 expression plasmids together (DBL). In this example, total protein extracted from Nicotiana benthamiana leaf tissue was analyzed by SDS-PAGE using stain-free technology (Bio-Rad, part 4568123) (FIGS. 14B, 14D, 14F), and by Western blot analysis with anti-FLAG (FIG. 14A), anti-HA (FIG. 14C), and anti-GFP (FIG. 14E) antibodies, with FLAG protein (FLG, lane 1), Positope (GFP, lane 2), and HA (HA, lane 3) included as positive controls.

Continuing this example, recombinant ovalbumin:GFP:FLAG was detected at the expected molecular weight of 74 kDA in plants transformed with the pMOZ183 expression plasmid alone and in plants co-transformed with the pMOZ183 and pMOZ246 expression plasmids together, but was not detected in wild type plants nor in plants transformed with the pMOZ246 expression plasmid alone (FIG. 14A).

Further continuing this example, recombinant ovomucoid:BFP:HA was detected at the expected molecular weight of 52 kDA in plants transformed with the pMOZ246 expression plasmid alone and in plants co-transformed with the pMOZ183 and the pMOZ246 expression plasmids together, but was not detected in wild type plants nor in plants transformed with the pMOZ183 expression plasmid alone (FIG. 14C).

Further continuing this example, recombinant ovalbumin:GFP:FLAG and recombinant ovomucoid:BFP:HA were detected at the expected molecular weights of 74 kDA and 52 kDA, respectively, in plants transformed with the pMOZ183 expression plasmid alone, plants transformed with the pMOZ246 expression plasmid alone, and plants co-transformed with the pMOZ183 and pMOZ246 expression plasmid together, but were not detected in wild type plants (FIG. 14E). These data show that the expected protein products ovalbumin:GFP:FLAG and ovomucoid:BFP:HA are produced in plants transformed with the pMOZ183 and pMOZ246 expression plasmids. Further, these data show that both ovalbumin:GFP:FLAG and ovomucoid:BFP:HA are produced in plants co-transformed the pMOZ183 and pMOZ246 expression plasmids together.

7. In Vivo Co-Localization of Ovalbumin and Ovomucoid Co-Expressed in Plants

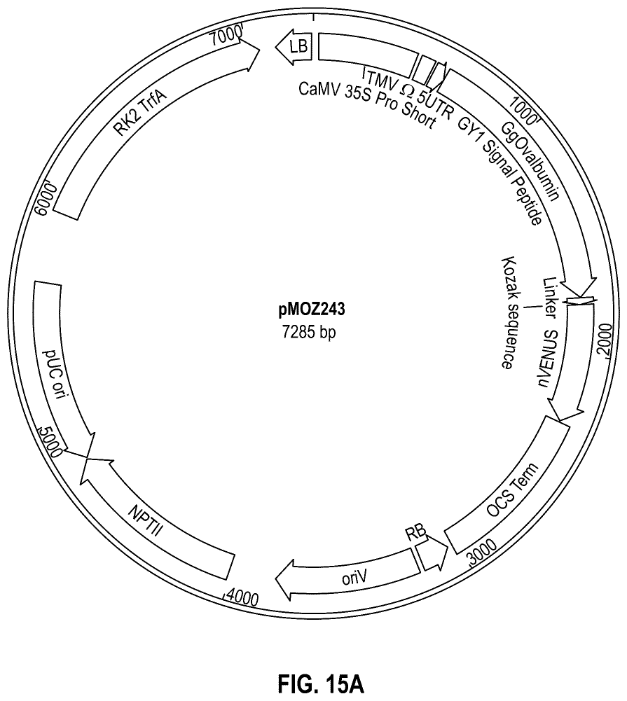

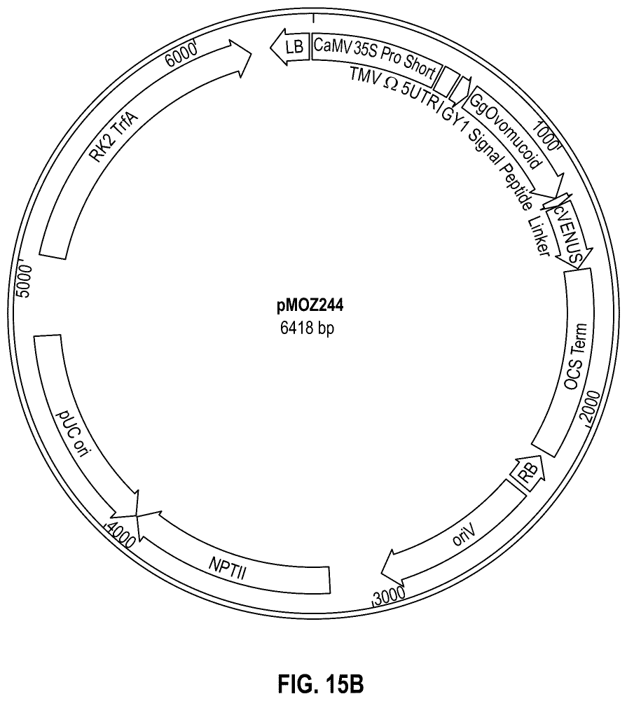

Referring now to FIG. 15A, therein is shown an example of a pMOZ expression plasmid for the production of recombinant Gallus gallus egg white proteins in plants constructed from the pMOZ vector backbone shown in FIG. 1. As a specific example, FIG. 15A is a map of a pMOZ243 expression plasmid [SEQ ID NO:35], which is an example expression plasmid for the production of recombinant ovalbumin:nVENUS in plants. Continuing this example, the pMOZ243 expression plasmid includes a 1158 bp target sequence encoding Gallus gallus ovalbumin (GgOvalbumin) [SEQ ID NO: 5] that had been synthesized using only coding sequences from the original Gallus gallus ovalbumin gene and was codon optimized for expression in plants using Nicotiana benthamiana as the model expression system, operatively linked to a 426 bp sequence encoding the promoter Cauliflower Mosaic Virus 35S promoter (CaMV 35S Pro Short) [SEQ ID NO:1], a 62 bp translational enhancer encoding the omega leader sequence of the Tobacco Mosaic Virus (TMV Ω 5UTR) [SEQ ID NO:2], a 57 bp N-terminal localization sequence encoding the soybean GY1 signal peptide (GY1 Signal Peptide) [SEQ ID NO:7] for localizing the recombinant protein to the endoplasmic reticulum of the plant cell where the recombinant protein can accumulate and not be degraded by the proteasome.

Further continuing this example, the pMOZ243 expression plasmid also includes a 519 bp label sequence encoding the N-terminal portion of the Aequorea victoria venus Yellow Fluorescent Protein gene (nVENUS) for analysis by bimolecular fluorescence complementation, a 30 bp linker sequence (Linker) to fuse the target sequence, a Kozak sequence [SEQ ID NO: 21] for strong initiation of translation, a 263 bp terminator sequence encoding octopine synthase terminator (OCS Term) [SEQ ID NO:4] that serves as a universal termination sequence for plant gene expression and can increase mRNA stability through polyadenylation, a right border repeat (RB) and left border repeat (LB) from nopaline C58 T-DNA [SEQ ID NO:27] for transgene integration into the plant host genome, a first origin of replication (pUC ori) [SEQ ID NO:24], a second origin of replication (oriV) [SEQ ID NO:25], an initiation sequence encoding the TrfA replication initiation protein (RK2 TrfA) [SEQ ID NO:26], and a selection sequence encoding neomycin phosphotransferase II (NPTII) [SEQ ID NO:23] encoding an aminoglycoside phosphotransferase conferring resistance to kanamycin.

Referring now to FIG. 15B, therein is shown an example of a pMOZ expression plasmid for the production of recombinant Gallus gallus egg white proteins in plants constructed from the pMOZ vector backbone shown in FIG. 1. As a specific example, FIG. 15B is a map of a pMOZ244 expression plasmid [SEQ ID NO:36], which is an example expression plasmid for the production of recombinant ovalbumin:cVENUS in plants according to the present disclosure. Continuing this example, the pMOZ244 expression plasmid includes a 558 bp target sequence encoding Gallus gallus ovomucoid (GgOvomucoid) [SEQ ID NO:6] that had been synthesized using only coding sequences from the original Gallus gallus ovomucoid gene and was codon optimized for expression in plants using Nicotiana benthamiana as the model expression system, operatively linked to a 426 bp sequence encoding the promoter Cauliflower Mosaic Virus 35S promoter (CaMV 35S Pro Short) [SEQ ID NO:1], a 62 bp translational enhancer encoding the omega leader sequence of the Tobacco Mosaic Virus (TMV Ω 5UTR) [SEQ ID NO:2], and a 57 bp N-terminal localization sequence encoding the soybean GY1 signal peptide (GY1 Signal Peptide) [SEQ ID NO:7] for localizing the recombinant protein to the endoplasmic reticulum of the plant cell where the recombinant protein can accumulate and not be degraded by the proteasome.

Further continuing this example, the pMOZ244 expression plasmid also includes a 252 bp label sequence encoding the C-terminal portion of the Aequorea victoria venus Yellow Fluorescent Protein gene (cVENUS) for analysis by bimolecular fluorescence complementation, a 30 bp linker sequence (Linker) to fuse the target sequence to the label sequence, a 263 bp terminator sequence encoding octopine synthase terminator (OCS Term) [SEQ ID NO:4] that serves as a universal termination sequence for plant gene expression and can increase mRNA stability through polyadenylation, a right border repeat (RB) and left border repeat (LB) from nopaline C58 T-DNA [SEQ ID 27] for transgene integration into the plant host genome, a first origin of replication (pUC ori) [SEQ ID NO:24], a second origin of replication (oriV) [SEQ ID NO:25], an initiation sequence encoding the TrfA replication initiation protein (RK2 TrfA) [SEQ ID NO:26], and a selection sequence encoding neomycin phosphotransferase II (NPTII) [SEQ ID NO:23] encoding an aminoglycoside phosphotransferase conferring resistance to kanamycin.

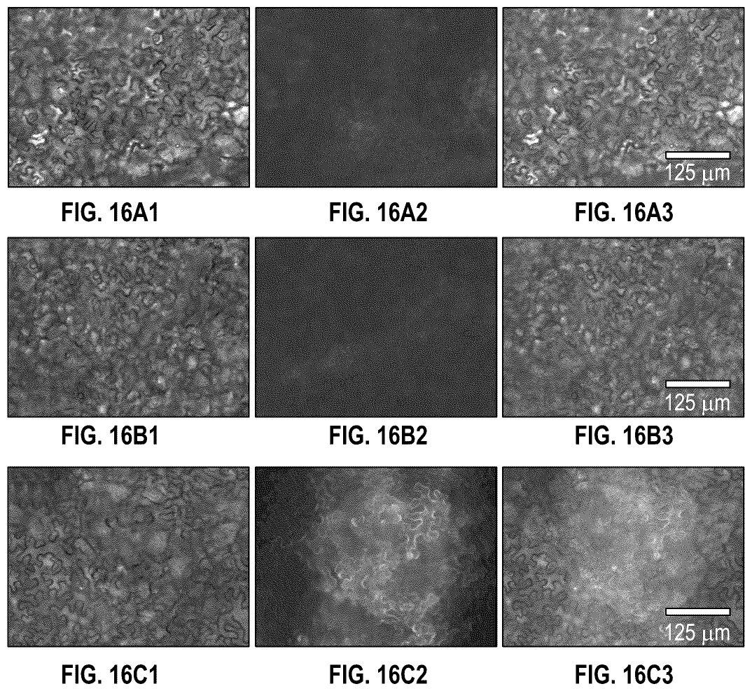

Referring now to FIGS. 16A1-16C3, therein is shown results from bi-molecular fluorescence complementation analysis of in vivo protein-protein interactions using the pMOZ243 expression plasmid [SEQ ID NO:35] in FIG. 15A and the pMOZ244 expression plasmid [SEQ ID NO:36] in FIG. 15B. In this example, Nicotiana benthamiana plants were incubated in a growth room at 25° C. with a 16 hour light 8 hour dark cycle for 4 weeks. The 4-week old Nicotiana benthamiana plants were infiltrated with Agrobacterium tumefaciens strain GV3101 carrying the pMOZ243 expression plasmid alone (FIGS. 16A1-16A3), with Agrobacterium tumefaciens strain GV3101 carrying the pMOZ244 expression plasmid alone (FIGS. 16B1-16B3), or a mixture of Agrobacterium tumefaciens strain GV3101 carrying the pMOZ243 expression plasmid and Agrobacterium tumefaciens strain GV3101 carrying the pMOZ244 expression plasmid (FIGS. 16C1-16C3). Following vacuum infiltration, plants were blot-dried and returned to the growth room for 72 hours before being imaged. Tissue sections from the systemically-infected Nicotiana benthamiana plants were visualized using an epifluorescence microscope.

Continuing this example, leaf epidermal cells were intact and turgid when viewed with an epifluorescence microscope by brightfield (FIGS. 16A1, 16B1, 16C1). When viewed with the Yellow Fluorescent Protein (YFP) excitation and emission filters, fluorescence was observed in leaf tissue sections from plants co-transformed with both the pMOZ243 and the pMOZ244 expression plasmids (FIG. 16C2), but was not observed in plants transformed with the pMOZ243 expression plasmid alone (FIG. 16A2) or with the pMOZ244 expression plasmid alone (FIG. 16B2), with similar results obtained in over 500 leaf tissue sections from 3 different plants. These data show that recombinant ovalbumin:nVENUS from the pMOZ243 expression plasmid and recombinant ovomucoid:cVENUS from the pMOZ244 expression plasmid are co-localized in leaf epidermal cells and suggest that these proteins interact in vivo.

8. Food Compositions

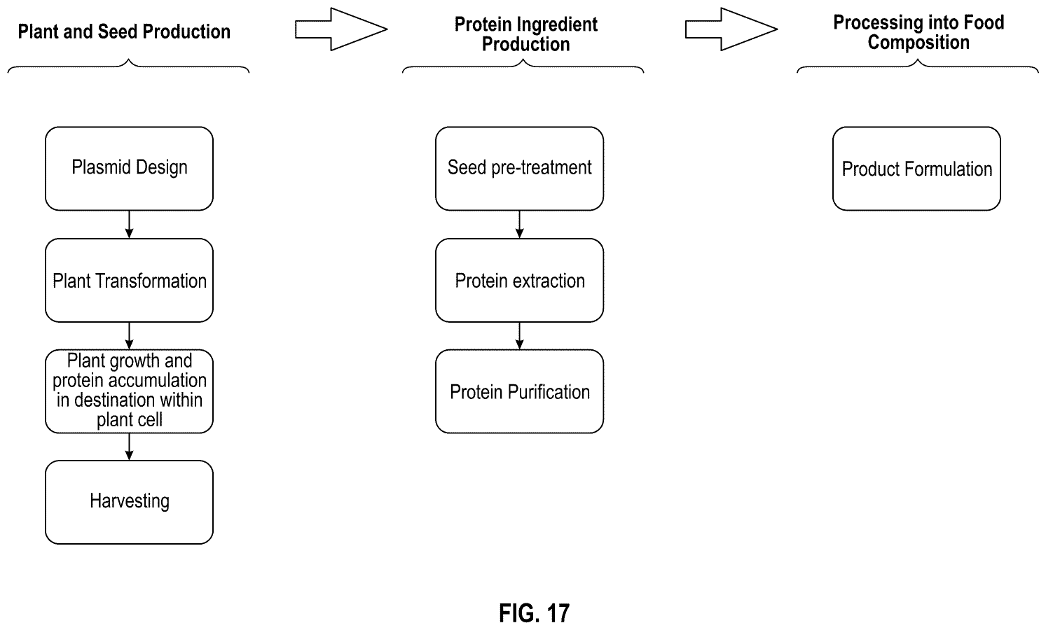

Referring now to FIG. 17, therein is shown an illustrative process for producing a food composition including a recombinant egg white protein produced in a plant. In this example, a plasmid is designed by cloning an expression cassette encoding an egg white protein into a pMOV vector backbone. The expression plasmid is propagated in bacteria and used to transform a plant that is grown to produce the egg white protein, which accumulates in a destination in a plant cell such as the apoplast, endoplasmic reticulum, nucleus, cytoplasm, protein storage vacuole or other vacuole. The transgenic plant is harvested, and the recombinant egg white protein is purified using extraction and purification methods. An egg white protein that is a tagged protein may be purified using affinity chromatography or other methods for purification of the tagged protein. The recombinant egg white proteins are then combined with other ingredients to produce food compositions such as egg and processed egg products, egg-substitutes, French toasts, frozen desserts, desserts, baked goods, toppings, icings, fillings, low-fat spreads, protein powders, protein bars, egg-based dry mixes, soups, sauces, salad dressings, geriatric nutrition, analog egg products, baby formula, infant formula, smoothies, butter alternatives, medical and clinical nutrition products, sports beverages, confections, meat products, analog meat products, meal replacement beverages, weight management food and beverages, protein powder, ice cream, marshmallow, marzipan, mayonnaise, meringue and meringue powders, pancake mixes, pastas, and specialty coffee drinks. Egg white proteins that are tagged proteins may include additional amino acids that could offer nutritional benefits by providing a more complete amino acid profile.

The resulting method, process, apparatus, device, product, and system is cost-effective, highly versatile, and accurate, and can be implemented by adapting components for ready, efficient, and economical manufacturing, application, production, and utilization. Another important aspect of an embodiment of the present disclosure is that it valuably supports and services the historical trend of reducing costs, simplifying systems, and increasing yield.

While some embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. An expression cassette for producing an egg white protein in a plant cell, comprising:
a target sequence encoding an egg white protein;
a promoter operatively linked to the target sequence;
a translational enhancer operatively linked to the target sequence; and
a terminator sequence operatively linked to the target sequence.

2. The expression cassette as claimed in claim 1, further comprising:
a polynucleotide sequence for localizing or retaining the egg white protein in a destination in a plant cell.

3. The expression cassette as claimed in claim 1, further comprising:
a localization sequence encoding a signal peptide for localizing the egg white protein to an endoplasmic reticulum in the plant cell.

4. The expression cassette as claimed in claim 1, wherein the signal peptide comprises sequences comprising at least 80% sequence identity to SEQ ID NO:7, SEQ ID NO:8, or SEQ ID NO:9.

5. The expression cassette as claimed in claim 1, further comprising a retention sequence encoding a peptide for retaining the egg white protein in an endoplasmic reticulum in the plant cell.

6. The expression cassette as claimed in claim 5, wherein the peptide comprises amino acid sequence comprises HDEL (SEQ ID NO: 37) or KDEL (SEQ ID NO: 38).

7-27. (canceled)

28. A plant stably transformed with the expression cassette of claim 1.

29. The plant of claim 28, wherein the plant is a dicot selected from the group consisting of an Arabidopsis, a tobacco, a tomato, a potato, a sweet potato, a cassava, an alfalfa, a lima bean, a pea, a chick pea, a soybean, a carrot, a strawberry, a lettuce, an oak, a maple, a walnut, a rose, a mint, a squash, a daisy, a quinoa, a buckwheat, a mung bean, a cow pea, a lentil, a lupin, a peanut, a fava bean, a French bean, a mustard, or a cactus.

30. The plant of claim 28, wherein the plant is a monocot selected from the group consisting of turf grass, corn, rice, oat, wheat, barley, sorghum, orchid, iris, lily, onion, palm, or duckweed.

31. The plant of claim 28, wherein at least 50% of leaf epidermal cells in the plant are transfected with the expression cassette and express at least one egg white protein.

32. The plant of claim 28, wherein the plant co-expresses at least two different egg white proteins.

33. The plant in claim 32, wherein the plant co-expresses ovomucoid and ovalbumin.

34-41. (canceled)

42. A food composition comprising an egg white protein produced using the plants as claimed in claim 28.

43. The food composition of claim 42, wherein the food composition is selected from the group consisting of an egg and a processed egg product, an egg-substitute, a French toast, a frozen dessert, a dessert, a baked good, a topping, an icing, a filling, a low-fat spread, a protein powder, a protein bar, an egg-based dry mix, a soup, a sauce, a salad dressing, a geriatric nutrition, an analog egg product, a baby formula, an infant formula, a smoothie, a butter alternative, a medical and clinical nutrition product, a sports beverage, a confection, a meat product, an analog meat product, a meal replacement beverage, a weight management food and beverage, protein powder, an ice cream, a marshmallow, a marzipan, a mayonnaise, a meringue and meringue powder, a pancake mix, a pasta, or a specialty coffee drink.

44-52. (canceled)

53. A food composition comprising:
a recombinant egg white protein, and
a plant protein.

54. The food composition in claim 53, wherein the plant protein is less than 5% (w/w) of total protein content in the composition.

55-72. (canceled)

73. The food composition in claim 53, wherein the food composition resembles an animal-derived egg white in at least one of color, browning, viscosity, density, rise/height, dome, spread, shape, hardness/firmness, adhesiveness, resilience/recoverable energy, structural integrity/cohesiveness, elasticity/springiness/rebound, chewiness/breakdown, taste, flavor, mouth-feel, or eating quality.

74. A method of producing a food composition, comprising,
growing a plant that expresses one or more recombinant egg white proteins;
harvesting a portion of the plant; and
isolating the recombinant egg white protein from the plant portion.

75. The method in claim 74, further comprising heating the food composition to cause the food composition to solidify.

76. The method in claim 74, wherein heating the composition is performed at a temperature between about 40° C. and about 200° C.

77-100. (canceled)
